(19) United States
(12) Patent Application Publication
(10) Pub. No.: US 2023/0233736 A1
(43) Pub. Date: Jul. 27, 2023

(54) ANTIMICROBIAL FORMULATIONS COMPRISING SILICONE

(71) Applicant: AMICOAT AS, Tromsø (NO)

(72) Inventors: Andreas Spiegelberg, Horgen (CH); Wenche Stensen, Kvaløysletta (NO); John Sigurd Svendsen, Kvaløysletta (NO)

(73) Assignee: AMICOAT AS, Tromsø (NO)

(21) Appl. No.: 18/001,382

(22) PCT Filed: Jun. 14, 2021
(86) PCT No.: PCT/EP2021/065998

§ 371 Date: Dec. 9, 2022

(30) Foreign Application Priority Data
Jun. 12, 2020 (GB) 2008978.5

(51) Int. Cl. A61L 15/58; A01N 63/50; A01N 25/10; A01P 1/00; A61L 15/44

(52) U.S. Cl. CPC A61L 15/585; A01N 63/50; A01N 25/10; A01P 1/00; A61L 15/44; A61L 2300/404

(57) ABSTRACT

The present invention provides a controlled release formulation comprising a silicone substrate which comprises a compound of Formula (I): AA-AA-AA-X—Y. The invention further provides methods of making these formulations, medical devices such as dressings incorporating said formulations and medical uses thereof.

The present invention relates to antimicrobial formulations, in particular to formulations comprising silicone and an antimicrobial small peptide or peptide-like molecule. These formulations may be used in or as antimicrobial articles, such as wound dressings, contact lenses and other medical devices such as implants, or as medical adhesives and adhesive patches.

Silicone elastomers, also known as polysiloxane elastomers, are a class of flexible, lightweight, thermally-stable and chemical-resistant polymers. Due to their favourable properties, silicone elastomers have a wide range of uses, including in medical devices, such as catheters and implants, for example in breast implants. Silicone elastomers are also used as medical adhesives and in wound dressings. Silicone hydrogels are used in contact lenses.

The use of medical devices such as catheters, orthopaedic devices and other implants has increased. Despite improvements in device design and surgical procedures, infections associated with such medical devices are still a major concern. Conventional antibiotic treatment often fails due to the low level of antibiotic at the actual site of infection and in the area surrounding the site of infection. The presence of biofilms on the introduced biomaterial/device can impair the efficacy of antibiotic treatment as can the existence of drug resistant strains.

Antimicrobial peptides (AMPs) are promising candidates as new antimicrobial agents as they are active against a broad spectrum of planktonic bacteria and biofilms, including antibiotic resistant strains. Moreover, bacteria are less likely to develop resistance to these rapidly acting peptides due to their mode of action, which includes disruption of the lipid membrane, rather than acting on a protein target.

Mishra et al. J. Mater. Chem. B, 2014, 2, 1706-1716 discloses silicone catheters coated with the AMP Lasioglossin-III. In this study the AMP was covalently immobilized to the silicone catheter surface. The treated catheter prevented biofilm growth of E. coli and E. faecalis and showed antimicrobial activity when submersed in phosphate buffered saline and synthetic urine over 4 days.

However, there remains a need for alternative formulations that may be used in medical devices which can act to provide controlled release of an antimicrobial agent as well as limiting colonisation of the device itself. To provide antimicrobial activity AMPs must also not be degraded, thermally or otherwise, during the manufacture of the formulation. Although silicone has many uses in medical devices, it cannot be assumed that antimicrobial peptides or peptide-like molecules can be combined with a silicone substrate in a way which provides controlled release of the antimicrobial peptide as well as limiting colonisation of the device itself. Formulating peptides into substrates which can provide controlled release is not straightforward, peptides are generally poorly soluble compared to other classes of pharmaceutical and are usually degraded at the kinds of temperatures which may be required for blending the peptide into a silicone substrate.

The present inventors have been able to prepare formulations which can be used to provide controlled release of an active antimicrobial agent, i.e. the antimicrobial agent is leachable and can inhibit bacterial growth in the surrounding environment. The antimicrobial agent also acts to control microbial growth within and on the surface of the formulation.

Thus, in one aspect, the present invention provides a controlled release formulation comprising (or consisting of) a silicone substrate which comprises a compound of formula (I)

AA-AA-AA-X—Y (I)

wherein, in any order, 2 of said AA (amino acid) moieties are cationic amino acids, preferably lysine or arginine but may be histidine or any non-genetically coded or modified amino acid carrying a positive charge at pH 7.0, and 1 of said AA is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms (in the case of fused rings of course the non-hydrogen atoms may be shared);

X is a N atom, which may be but preferably is not substituted by a branched or unbranched C₁-C₁₀ alkyl or aryl group, e.g. methyl, ethyl or phenyl, and this group may incorporate up to 2 heteroatoms selected from N, O and S; and

Y is selected from the group consisting of R₁—R₂—R₃,

R₁—R₂—R₂—R₃, R₂—R₂—R₁—R₃, R₁—R₃ and R₄

wherein:

R₁ is C, O, S or N, preferably C;

R₂ is C;

each of R₁ and R₂ may be substituted by C₁-C₄ alkyl groups or unsubstituted, preferably Y is —R₁—R₂—R₃ (in which R₁ is preferably C) and preferably this group is not substituted, when Y is —R₁—R₂—R₂—R₃ or R₂—R₂—R₁—R₃ then preferably one or more of R₁ and R₂ is substituted;

R₃ is a group comprising 1 to 3 cyclic groups each of 5 or 6 non-hydrogen atoms (preferably all C atoms but optionally also containing N, O or S), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; R₃ incorporates a maximum of 15 non-hydrogen atoms, preferably 5-12, most preferably it is phenyl; and

R₄ is an aliphatic moiety having 2-20 non-hydrogen atoms, preferably these are carbon atoms but oxygen, nitrogen or sulphur atoms may be incorporated, preferably R₄ comprises 3-10, most preferably 3-6 non-hydrogen atoms and the moiety may be linear, branched or cyclic. If the R₄ group comprises a cyclic group this is preferably attached directly to the nitrogen atom of X.

Preferred compounds incorporate an R₄ group which is linear or branched, in particular a linear or branched alkyl group including ethyl, propyl, isopropyl, butyl, isobutyl, sec-butyl, tert-butyl, pentyl and isomers thereof, hexyl and isomers thereof etc.; propyl, isopropyl, butyl and isobutyl are especially preferred.

In some embodiments, R₄ is an aliphatic moiety (preferably an alkyl group) having 6-16 non-hydrogen atoms, preferably these are carbon atoms but oxygen, nitrogen or sulphur atoms may be incorporated, and the moiety may be linear, branched or cyclic.

In some preferred embodiments, R₄ is an isopropyl group.

Of the R₄ groups which comprise a cyclic group, preferred are molecules in which R₄ is cyclohexyl or cyclopentyl.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethyllysine and trimethylornithine, 4-aminopiperidine-4-carboxylic acid, 4-amino carbamimidoylpiperidine-4-carboxylic acid and 4-guanidinophenylalanine.

The large lipophilic R group of the AA may contain hetero atoms such as 0, N or S, typically there is no more than one heteroatom, preferably it is nitrogen. This R group will preferably have no more than 2 polar groups, more preferably none or one, most preferably none.

The compounds, which are preferably peptides, are preferably of formula (II)

AA₁-AA₂-AA₁-X—Y (II)

wherein:

AA₁ is a cationic amino acid, preferably lysine or arginine but may be histidine or any non-genetically coded or modified amino acid carrying a positive charge at pH 7.0;

AA₂ is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms; and

X and Y are as defined above.

Further preferred compounds include compounds of formulae (III) and (IV):

AA₂-AA₁-AA₁-X—Y (III)

AA₁-AA₁-AA₂-X—Y (IV)

wherein AA₁, AA₂, X and Y are as defined above. Molecules of formula (II) are more preferred.

From amongst the above compounds certain are particularly preferred. In particular, compounds wherein the amino acid with a large lipophilic R group, conveniently referred to herein as AA₂, is tributyl tryptophan (Tbt) or a biphenylalanine derivative such as Phe (4-(2-Naphthyl)) [also referred to herein as Bip (4-(2-Naphthyl)], Phe (4-(1-Naphthyl)) [also referred to herein as Bip (4-(1-Naphthyl)], Bip (4-n-Bu), Bip (4-Ph) or Bip (4-T-Bu); Phe (4-(2-Naphthyl)) and Tbt being most preferred. In some preferred embodiments, the amino acid with a large lipophilic R group is tributyl tryptophan (Tbt).

Another preferred group of compounds are those wherein Y is —R₁—R₂—R₃ as defined above, preferably wherein R₁ and R2 are unsubstituted, most preferably wherein R₁ and R2 are both carbon atoms.

A further preferred group of compounds are those in which —X—Y together is the group —NHCH₂CH₂Ph.

The compounds include all enantiomeric forms, both D and L amino acids and enantiomers resulting from chiral centers within the amino acid R groups and the C-terminal capping group “—X—Y”. β and γ amino acids as well as a amino acids are included within the term ‘amino acids’, as are N-substituted glycines which may all be considered AA units. The molecules of the invention include beta peptides and depsipeptides.

Most preferred compounds are the following:

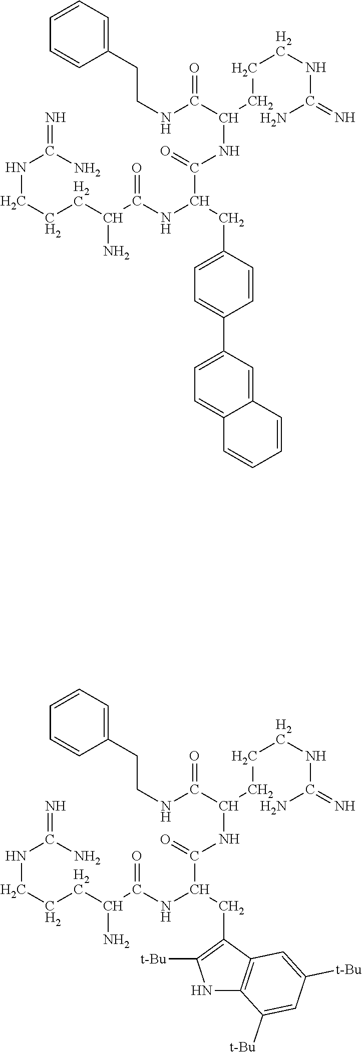 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1 ; CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

t-Bu represents a tertiary butyl group. This second compound incorporating the amino acid 2,5,7-Tris-tert-butyl-L-tryptophan is the most preferred compound of use in the present invention (and is also referred to herein as AMC-109). Analogues of this compound incorporating other cationic residues in place of Arg, in particular Lys, are also highly preferred. Analogues incorporating alternative C terminal capping groups as defined above are also highly preferred.

A further preferred group of compounds are those in which —X—Y together is selected from the group consisting of —NHCH(CH₃)₂, —NH(CH₂)₅CH₃, —NH(CH₂)₃CH₃, —NH(CH₂)₂CH₃, —NHCH₂CH(CH₃)₂, —NHcyclohexyl and —NHcyclopentyl, particularly preferred are compounds in which —X—Y is the group —NHCH(CH₃)₂ or —NH(CH₂)₅CH₃. A particularly preferred group of compounds are those in which —X—Y together is NHCH(CH₃)₂.

A preferred compound is a compound in which AA₁ is arginine, AA₂ is tributyl tryptophan and —X—Y together is NHCH(CH₃)₂.

Compounds of use in the present invention are preferably peptides.

The compounds of formulae (I) to (IV) may be peptidomimetics and peptidomimetics of the peptides described and defined herein also represent compounds of use in accordance with of the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the molecule is reacting with a membrane rather than the specific active site of an enzyme, some of the problems described of exactly mimicking affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds of the present invention will typically have 3 identifiable sub-units which are approximately equivalent in size and function to amino acids (AA units). The term ‘amino acid’ may thus conveniently be used herein to refer to the equivalent sub-unit of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

Compounds (e.g. peptides) of use in accordance with the present invention exhibit antimicrobial activity (typically antibacterial activity), in particular they exert a cytotoxic effect through a direct membrane-affecting mechanism and can be termed membrane acting antimicrobial agents. These compounds are lytic, destabilising or even perforating the cell membrane. This offers a distinct therapeutic advantage over agents which act on or interact with proteinaceous components of the target cells, e.g. cell surface receptors. While mutations may result in new forms of the target proteins leading to antibiotic resistance, it is much less likely that radical changes to the lipid membranes could occur to prevent the cytotoxic effect. The lytic effect causes very rapid cell death and thus has the advantage of killing bacteria before they have a chance to multiply. In addition, the molecules may have other useful properties which kill or harm the target microbes e.g. an ability to inhibit protein synthesis, thus they may have multi-target activity.

The compounds for use in the invention may be synthesised in any convenient way. Generally the reactive groups present (for example amino, thiol and/or carboxyl) will be protected during overall synthesis. The final step in the synthesis will thus be the deprotection of a protected derivative of the invention.

In building up a peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the α-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPCIP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed. The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

The silicone substrate is formed predominantly (at least 50%, 60%. 70%, 80% or 90%) or entirely (at least 99%) by weight of silicone. The silicone present in the formulation may be of a single or multiple different types. Silicones may be referred to in the art and herein as polysiloxanes.

Silicones or polysiloxanes are polymers which are made up of repeating units of siloxane, which is a chain of alternating silicon atoms and oxygen atoms, combined with carbon, hydrogen, and sometimes other elements. Thus, polysiloxanes contain an inorganic silicon-oxygen backbone chain ( . . . —Si—O—Si—O—Si—O— . . . ) with organic side groups attached to the silicon atoms such that each silicon atom is tetravalent. Linear silicones can therefore be represented by the general chemical formula [R₂SiO₂]n, where R is an organic group or hydrogen and n is an integer greater than 1. The R groups in each monomer submit may be the same or different. Branched silicones may also be used.

By varying the —Si—O— chain lengths and the nature of the organic side groups, silicones can be synthesized with a wide variety of properties and compositions. Preferred R groups include hydrogen, methyl, ethyl, propyl and phenyl, which may optionally be substituted, e.g. with a halogen such as fluorine. Thus suitable polymers may comprise dimethyl and diphenyl monomer subunits. Silicones may preferably be vinyl-terminated.

The silicone is preferably a silicone elastomer. The silicone will typically be a medical grade silicone elastomer. A variety of different silicone based materials and substrates are known in the art which have utility as medical and other devices which are in contact with the human or animal body. The following types of silicone are suitable for use in accordance with the present invention: silicone sealants/adhesives, liquid silicone rubber (LSR, mainly used for injection moulded products), silicone coatings/dispersions, silicone foams, silicone membranes, silicone fluids and gels (for example used in breast implants), high consistency silicone rubbers (mainly used for extruded products), low viscosity elastomers, pressure sensitive adhesives (PSA) and tacky gels. Any of these may be combined with a compound of formula (I).

Polysiloxane or silicone elastomers can be formed by cross-linking individual polymer chains to form a 3D network. The process of crosslinking polysiloxanes to form polysiloxane elastomers may be termed curing or vulcanising. Curing may take place in the presence of the compound of formula (I), e.g. with an impregnated silicone PSA, but will not in other production methods. Curing may advantageously take place in the presence of a platinum catalyst which, surprisingly, does not have a detrimental effect on the compound of formula (I).

The silicone is preferably a cured silicone elastomer, curing may involve heating to a temperature in the range of about 100-200° C. for about 1-5 hours, optionally in the presence of a suitable catalyst. In other embodiments, curing may involve drying at ambient temperature (e.g. for 12 or more or 24 or more hours). Suitable catalysts include platinum, palladium and peroxide based catalysts. A condensation curing system may be employed.

Suppliers of suitable silicones include Avantor/Nusil and Dow/DuPont.

Some preferred silicone substrates contain a solvent which can be used to dissolve the compound of formula (I), or (more usually) is miscible with a solvent in which the compound of formula (I) has been dissolved, e.g. silicones which are PSAs. Flowable silicone products such as PSAs can be applied to a substrate by dipping or painting.

Foam silicone substrates may be used according to the present invention, for example the wound dressings sold under the name Mepilex Lite by Molynlycke Healthcare or Allevyn Gentle Border sold by Smith & Nephew. Such products with preformed cavities can take up a liquid in which is dissolved a compound of formula (I), e.g. ethanol or water. They can behave like sponges, taking up the liquid and thus the dissolved compound penetrates into the material. The foam may swell but typically returns to its normal size as it dries; the solvent evaporates but the compound remains dispersed through the silicone foam.

Harder silicones are used in other applications, e.g. to provide solid medical devices, or components therefor, which are not flowable products like the adhesives. Harder silicones include the preferred Nusil MED-4065 and LSR. Harder silicones may be formed in the presence of the active agent such that the active agent is compounded throughout the silicone substrate.

A preferred silicone elastomer of the harder type is formed by mixing a compound of formula (V):

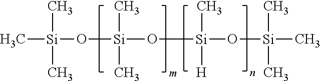

with an organopolysiloxane having silicon-bonded alkenyl groups and optionally an inorganic filler and then curing the resultant mixture (curable silicone elastomer composition) under suitable curing conditions to form the silicone elastomer. “m” is preferably larger than “n”, e.g. at least 2, 3, 4 or 5 times larger. The polymer is classified as being of high molecular weight.

The compound of formula (V) is referred to as methylhydrosiloxane, dimethylsiloxane copolymer, trimethylsiloxane terminated (and sometimes as Siloxanes and Silicones, dimethyl, methyl hydrogen) (CAS-No. 68037-59-2).

The present inventors have developed a number of different ways in which the silicone and compound of formula (I) may be combined.

According to the present invention, the silicone may be compounded with the compound of formula (I) by mixing the compound of formula (I) with a compound of formula (V), an organopolysiloxane having silicon-bonded alkenyl groups and, optionally, an inorganic filler. The order for combining the compound of formula (V) with the other components is not limited. For example, the compound of formula (I) can be mixed with one of the components of the curable silicone elastomer composition, such as the compound of formula (V), and then combined with the other components of the curable silicone elastomer composition. The silicone is cured after the compound of formula (I) has been added thereto. Such methods are described in the Examples and are further aspects of the present invention.

Kits comprising a compound of formula (V), an organopolysiloxane having silicon-bonded alkenyl groups and a platinum catalyst are available commercially, for example from Avantor® under the trade name Nusil™ MED-4065, which forms a medical grade silicone elastomer after curing. MED-4065 is a firm silicone, softer silicones in the Nusil range which may be used include MED-4035 and MED-4050.

The components may be mixed using conventional methods that are well known in the art, for example using a two roll mill.

The curing conditions may comprise heating to a temperature in the range of about 100-200° C. Curing times will typically be for about 1-5 hours, in the presence of a suitable catalyst. Preferably, the curing conditions comprise heating to a temperature in the range of about 120-180° C. for about 1-5 hours. More preferably, the heat curing reaction comprises heating to a temperature in the range of about 130-150° C. for about 2-4 hours. Suitable catalysts are platinum catalysts.

The silicone elastomer may have a hardness, measured in accordance with ASTM D2240 using a type A durometer hardness tester (Shore-A hardness) of from 25-90, more preferably 40-80, most preferably from 50-70. Preferably, the silicone elastomer has a tensile strength, measured in accordance with ASTM D412, of from about 5-20 MPa, more preferably from about 6-15 MPa, most preferably about 8-10 MPa. Preferably, the silicone elastomer has a breaking elongation of from about 800-1300%, more preferably from about 900-1100%. These preferred characteristics may be combined in any manner.

Further suitable silicone elastomers for use in the present invention are of the type disclosed in US2016369100 A, the disclosure of which is incorporated herein by reference. These silicones are of particular utility as medical devices requiring a firm silicone such as an implant, catheter or suture.

These silicone elastomers may be formed by curing a curable silicone elastomer composition comprising:

(A) an organopolysiloxane having silicon-bonded alkenyl groups;
(B) an organohydrogensiloxane having an average of two or more silicon-bonded hydrogen atoms in the molecule;
(C) an inorganic filler; and optionally
(D) at least one, and more preferably a mixture of, filler treatment agents.

Curing may take place in the presence of a compound of formula (I).

The composition can further include (E) a catalytically effective amount of an addition reaction catalyst or the catalyst can be provided to the composition including components (A) through (D) at a later time. When the composition includes the catalyst (E), it is preferable to further include a curing retarder (i.e., inhibitor).

The (A) organopolysiloxane preferably has a number-average degree of polymerization of 2,000 or greater based on the number-average molecular weight in terms of standard polystyrene equivalent as measured by gel permeation chromatography (GPC) (hereinafter referred to as “number-average degree of polymerization”), and which exhibits a raw rubber state or gum state at room temperature. Preferably, the number-average degree of polymerization is of (A) is from 2,000 to 100,000, more preferably from 3,000 to 8,000.

The (A) organopolysiloxane can include siloxane units having hydrocarbon groups R, (e.g., —SiOR₂—) where each R can be the same or different and are substituted or unsubstituted monovalent hydrocarbon groups. Such R groups can have from 1 to 10 carbons, and preferably from 1 to 8 carbons. Examples of the R include alkyl groups such as methyl groups, ethyl groups, propyl groups, isopropyl groups, butyl groups, isobutyl groups, tert-butyl groups, pentyl groups, neopentyl groups, hexyl groups, cyclohexyl groups, octyl groups, nonyl groups and decyl groups; aryl groups such as phenyl groups, tolyl groups, xylyl groups and naphthyl groups; aralkyl groups such as benzyl groups, phenylethyl groups and phenylpropyl groups; alkenyl groups such as vinyl groups, allyl groups, propenyl groups, isopropenyl groups, butenyl groups, hexenyl groups, cyclohexenyl groups and octenyl groups; groups obtained by substituting some or all of the hydrogen atoms in the aforementioned groups with halogen atoms such as fluorine atoms, bromine atoms and chlorine atoms, cyano groups and the like, such as chloromethyl groups, chloropropyl groups, bromoethyl groups, trifluoropropyl groups and cyanoethyl groups, but it is preferable for 90% or more of the R groups to be methyl groups

The content of silicon-bonded alkenyl groups in component (A) is determined according to the degree of polymerization and the presence/absence of branches on the main chain, but component (A) is preferably a straight chain or partially branched organopolysiloxane in which the alkenyl group content is from 0.001 to 0.1 weight %, and more preferably a straight chain organopolysiloxane having an average of two or more silicon-bonded alkenyl groups at both molecular termini.

In one embodiment, the structure of component (A) is such that the molecular termini are capped by triorganosiloxy groups having silicon-bonded alkenyl groups and the main chain has a straight chain structure comprising repeating diorganosiloxane units, but may be a partially branched chain structure. The molecular weight of component (A) is such that the number-average degree of polymerization is 2,000 or greater (from 2,000 to 100,000) and component (A) exhibits in a raw rubber state or gum state, and the number-average degree of polymerization is preferably 3,000 or greater (from 3,000 to 8,000). If the number-average degree of polymerization is less than the aforementioned lower limit, it is difficult to obtain a satisfactory rubbery feeling, the surface may become sticky or tacky.

Component (B) is a crosslinking agent for the composition. The bonding sites of the silicon-bonded hydrogen atoms in component (B) are not particularly limited, and may be molecular termini, or pendant to (along) the molecular chains or molecular termini and pendant to the molecular chains. In addition, examples of silicon-bonded groups other than hydrogen atoms in component (B) include monovalent hydrocarbon groups, for example, alkyl groups such as methyl groups, ethyl groups, propyl groups, butyl groups, pentyl groups and hexyl groups; cycloalkyl groups such as cyclopentyl groups and cyclohexyl groups; aryl groups such as phenyl groups, tolyl groups and xylyl groups; aralkyl groups such as benzyl groups and phenethyl groups; halogenated alkyl groups such as 3,3,3-trifluoropropyl groups and 3-chloropropyl groups; alkenyl groups such as vinyl groups, allyl groups, propenyl groups, isopropenyl groups, butenyl groups, hexenyl groups, cyclohexenyl groups and octenyl groups, with alkyl groups and aryl groups being preferred and methyl groups and phenyl groups being particularly preferred.

The molecular structure of component (B) is not limited, and may be, for example, straight chain, branched chain, straight chain having some branches, cyclic, dendritic (dendrimer-like) or resin-like. Component (B) may be a homopolymer having these molecular structures, a copolymer comprising these molecular structures or a mixture thereof.

Component (C) is an inorganic filler, which imparts the silicone elastomer with strength, among other properties. It is possible to use one or more types of inorganic filler, and component (C) can be a reinforcing filler such as a silica fine powder or fumed titanium oxide; a non-reinforcing filler such as diatomaceous earth, an aluminosilicate, iron oxide, zinc oxide or calcium carbonate; or a thermally conductive filler such as aluminum oxide or boron nitride.

Component (D) is a surface treatment agent for the inorganic filler which includes an alkenyl-containing group. Examples of such a filler treatment agent include an alkenyl-containing organosilane, organosilazane, organosilanol, alkoxyorganosilane, or any combination thereof.

The addition reaction catalyst of component (E) is a catalyst used to facilitate curing of the present composition, and may be a platinum-based catalyst, a palladium-based catalyst, a rhodium-based catalyst and the like. A platinum metal-type catalyst is particularly preferred.

Examples of component (E) include platinum-based catalysts, for example, platinum fine powders, platinum black, chloroplatinic acid, platinum tetrachloride, alcohol-modified chloroplatinic acid, olefin complexes of platinum, alkenylsiloxane complexes of platinum, carbonyl complexes of platinum, carbene complexes of platinum, platinum on finely divided solid supports such as silica, powdered thermoplastic organic resins and silicone resins containing these platinum-based catalysts; rhodium-based catalysts, palladium based catalysts, other transition metal based catalysts.

The addition-curable silicone elastomer composition may contain a curing retarder in order to adjust the curing speed or pot life. Examples of curing retarders include alcohol derivatives having carbon-carbon triple bonds, such as 3-methyl butyn-3-ol, 3,5-dimethyl-1-hexyn-3-ol, phenylbutynol and 1-ethynyl-1-cyclohexanol; ene-yne compounds such as 3-methyl-3-penten-1-yne and 3,5-dimethyl-3-hexen yne; alkenyl group-containing low molecular weight siloxanes such as tetramethyltetravinylcyclotetrasiloxane and tetramethyltetrahexenylcyclotetrasiloxane; and alkyne-containing silanes such as methyl-tris(3-methyl-1-butyne-3-oxy)silane and vinyl-tris(3-methyl-1-butyne-3-oxy)silane.

The curable silicone elastomer composition can be prepared by homogeneously mixing components (A) through (E) together along with any optional ingredients. Mixing the components and ingredients can be achieved by any conventional means such as a Morehouse Cowles mixer, a two roll mill or a kneader mixer. The curable silicone elastomer composition may also comprise the compound of formula (I).

As an alternative to curing the silicone in the presence of a compound of formula (I), the inventors have developed a method of introducing the compound of formula (I) into the silicone substrate after it has been cured. This is achieved by the technique of swelling and drying whereby a solvent carrier in which the compound of formula (I) has been dissolved is used to impregnate the silicone substrate. The same harder silicones as described above may be used in this method. A silicone substrate is made or selected and an impregnation step via swelling is performed to introduce the compound of formula (I) into the silicone substrate. This method may be performed on a pre-formed silicone material or device.

The so-called “swell-dry” technique is described in the Examples and has the advantage that, in some embodiments, only the outer part of the substrate, device or article contains the active agent and so the product is cheaper to make without compromising efficacy.

A further type of silicone of use in medical devices is as a Pressure Sensitive Adhesive (PSA). These silicones are much softer and tackier than the silicones described above but are also suitable to be combined with a compound of Formula (I) to provide a formulation of the invention.

They may comprise a high molecular weight polydimethylsiloxane and a tackifying silicone resin dispersed in a solvent. Curing may be achieved using a platinum, palladium or peroxide catalyst. Prior to curing with a peroxide catalyst the solvent may be removed by heating at a moderate temperature and then increasing the temperature to cure and finally decompose the peroxide catalyst.

Curing preferably takes place after the compound of Formula (I) has been added, e.g. by air drying for several hours. For example, the PSA plus compound mixture is applied to a substrate (e.g. a medical device of interest) and then cured through a slow drying process to leave a tacky layer on the substrate.

The PSA may be applied on one or both sides of a substrate, such as a Kapton or Mylar, a foam or rubber or directly onto a release film.

An alternative to a PSA which also provides a sticky product is a tacky silicone gel; the basic siloxane polymer chemistry is the same but the gel lacks the silicone resin. These silicone gels are typically composed of two types of siloxane polymers: vinyl-functional polysiloxanes and hydride-functional polysiloxanes. These silicone gels are low-viscosity materials that are not dispersed in solvent systems. These silicone gels cure in the presence of platinum catalysts to solid forms that do not flow; gels can be formulated to cure completely at low temperatures. Commercially available examples include MED-6340, a dimethylpolysiloxane and GEL-9502-30, a diphenyldimethylpolysiloxane. These products have lower peel strength and higher surface tack than silicone PSA products.

Other silicone gels are not tacky and may be suitable for use in breast implants. These gels may have a viscosity of 100 centipoise to 100,000 centipoise but preferably about 1000 centipoise. The silicone gel may be supplied as a 2-part system where a Part A contains the vinyl-endblocked dimethyl siloxane polymer and platinum catalyst. Part B contains the vinyl endblocked dimethyl siloxane polymer, methyl-hydrogen crosslinker, and a suitable inhibitor such as methylvinylcyclosiloxane. The reactive siloxane polymer can have terminal vinyl groups pendant vinyl groups, or a combination of both. The siloxane polymer should have dimethyl substituted groups along the backbone but can also have diphenyl, methylphenyl, and trifluoropropyl substitution. The crosslinker can possess terminal hydride groups, pendant hydride groups, or a combination of both. The hydride concentration can range from 10 to 80 mole % but preferably 50 mole %. The catalyst should be platinum based at a concentration of 2 to 10 parts per million but preferably 8 ppm. Other catalysts can be iridium, palladium, rhodium, and other suitable catalysts. Examples of suitable materials include Nusil MED-6342, Nusil MED-6345, Nusil MED-6350, Nusil MED-6311 (NuSil Technology LLC, Carpinteria, Calif.) and Applied Silicone 40022, Applied Silicone 40135, and Applied Silicone 40008 (Applied Silicone Corporation, Santa Paula, Calif.). Silicone-based gels are particularly suitable materials for breast implants.

The formulations of the invention may comprise the compound of formula (I) in an amount of from about 0.005-10%, preferably 0.1-5%, in some embodiments 0.5-5% by weight based on the total weight content of solid matter. The % of the compound of formula (I) will tend to be lower where the compound is not dispersed throughout the silicone substrate but is present only at the periphery, for example as may be achieved by the swelling/drying approach.

For the avoidance of doubt, it is noted that there is no covalent attachment between the silicone and the compound of formula (I) in the formulations of the invention. Thus, the compound may be considered to be releasably associated with the silicone.

The compound of formula (I) is capable of being released from (or leaching from or diffusing out of) the formulation of the invention. This is important in the context of the present invention as compounds of formula (I) have antimicrobial activity, and it is desirable that in use the compounds are capable of being released from the formulation to an area that requires an antimicrobial activity, for example to prevent or treat an infection of a wound, or site of an implantation of a medical device.

Preferably, in use, there is controlled (i.e. sustained) release of the compound of Formula (I) from the formulation. For example, there may be release of a therapeutically effective amount of the compound for at least 6, 12, 24, 36, 48, 72 hours. A therapeutically effective amount will preferably result in delivery to the local environment of a concentration of the compound which is in excess of the Minimum Inhibitory Concentration (MIC) of the compound for the target bacteria.

The ability of active compound to be released from the formulation of the invention may be readily determined by any suitable method, and the skilled person is familiar with such methods. Suitable methods are described in the examples hereto. For example, a formulation of the present invention can be brought into contact with an agar plate that has been inoculated with bacteria (e.g. a bacteria of the genus Staphylococcus) and, after an appropriate incubation time, the plates can be inspected for the presence of a “zone of inhibition” (i.e. a zone with no bacterial growth or with reduced bacterial growth) around the suture. The presence of a “zone of inhibition” (e.g. as compared to a test with a formulation comprising silicone that does not contain an antimicrobial compound) is indicative that compound can be released from the formulation. Alternatively, extraction methods may be performed as described in the Examples.

A compound of formula (I) may be considered to be dispersed (releasably dispersed) through the silicone. This may be a homogenous dispersion through the entire silicone substrate or it may be in just one or more zones, e.g. in the outer portions of the substrate. For example, based on a cut cross section through the silicone substrate, 2-100% or 2-90%, preferably 5-75%, more preferably 5-50% or 5-30%, e.g. 5-20% or 10-20% of the silicone contains a compound of formula (I) dispersed therein.

In another aspect, the present invention provides a method comprising mixing a silicone substrate, or the component parts which form the silicone substrate, with a compound of formula (I) and optionally curing said mixture to provide a formulation of the invention. Preferably, when the (elastomeric) silicone substrate is formed by combining two components, this combination is performed first and then the compound of formula (I) is added thereto and then the whole mixture is cured. Embodiments of other aspects of the invention described herein apply, mutatis mutandis, to this aspect of the invention. The components may be mixed using conventional methods that are well known in the art, for example using a two roll mill. The curing conditions may comprise heating to a temperature in the range of about 100-200° C. Curing times will typically be for about 1-5 hours, in the presence of a suitable catalyst. Preferably, the curing conditions comprise heating to a temperature in the range of about 120-180° C. for about 1-5 hours. More preferably, the heat curing reaction comprises heating to a temperature in the range of about 130-150° C. for about 2-4 hours. Suitable catalysts are platinum catalysts.

The present invention provides a method of producing a formulation comprising a silicone substrate compounded with a compound of formula (I), which method comprises curing a curable silicone elastomer composition into which a compound of formula (I) has been mixed under suitable curing conditions to provide a silicone substrate compounded with the compound of formula (I). Embodiments of other aspects of the invention described herein apply, mutatis mutandis, to this aspect of the invention.

In a further aspect the present invention provides a method of producing a formulation of the invention, which method comprises (i) preparing a solution of one or more solvents and a compound of formula (I) dissolved therein, and (ii) applying said solution to a silicone substrate. The silicone substrate may be any of the types described herein, e.g. a foam, a PSA or a pre-formed (resilient) silicone product. In some preferred embodiments the silicone substrate will have been cured prior to step (ii) but the method may include a step (iii) of curing the product of step (ii), i.e. the silicone substrate to which the compound of formula (I) dissolved in solvent has been applied. A curing step (iii) will typically be employed when the silicone employed in step (ii) is a flowable silicone as used to make a PSA. Curing is typically by drying in this scenario.

For formulations where no curing step (iii) is performed there will still typically be a step (which may be considered step (iii a)) of drying the product of step (ii). Air drying at room temperature is preferred but standard methods to accelerate drying may be used, e.g. moderate heating.

If the silicone substrate is a foam then the solvent(s) employed in step (i) only needs to be able to dissolve the compound of formula (I) as the solvent(s) can carry the compound into the cavities within the silicone foam. Thus a single solvent such as ethanol or even water may be employed. It is a greater challenge to introduce the compound into non-foam silicone substrates. For those products the solvent(s) used in step (i) must be able to dissolve the compound of Formula (I) and; either (a) be compatible with a solvent already present in the silicone substrate to allow mixing of the compound and the silicone, for example in the case of flowable silicone products such as those used to make a PSA; or (b) comprise a compatible solvent mixture (typically two solvents) which is able to dissolve the compound and penetrate the silicone and cause the silicone to swell, allowing the compound of formula (I) to be dispersed within the silicone.

In a further aspect the present invention provides a method of producing a formulation of the invention which comprises a silicone adhesive (a silicone PSA), which method comprises dissolving a compound of formula (I) in a first solvent and mixing with an adhesive silicone containing formulation which also comprises a solvent which is the same as or miscible with said first solvent. Said mixture may be cooled to a temperature below 20 degrees C., e.g. to a temperature between 2.5 and 15 degrees C., preferably to a temperature between 5 and 10 degrees C. to ensure homogeneity. Said formulation may optionally be applied to a backing material, fabric, foam, rubber or device or other substrate. Said formulation may be cured, e.g. by leaving it to dry at ambient temperature for at least 6 hours, e.g., at least 8 hours, e.g. 10-16 hours.

As discussed above, the inventors have also developed a method of introducing the compound of formula (I) into the silicone substrate after it has been cured. This is conveniently achieved by the technique of swelling and drying whereby a solvent carrier in which the compound of formula (I) has been dissolved is used to impregnate the silicone substrate. A silicone substrate is made or selected and an impregnation step via swelling is performed to introduce the compound of formula (I) into the silicone substrate. This method may be performed on a pre-formed silicone material or device.

In a preferred method according to the present invention the formulation of the invention is prepared by applying a solution of a compound of formula (I) to a silicone substrate and then allowing the silicone to dry. Typically, after application of the solution of a compound of formula (I) the silicone swells (as it absorbs the solution).

Thus, in another aspect, the present invention provides a method of producing a silicone substrate impregnated with a compound of formula (I), said method comprising (i) applying a solution of a compound of formula (I) to the silicone and (ii) drying the silicone to which said solution has been applied, thereby producing a silicone substrate impregnated with a compound of formula (I). Typically, the applying of step (i) leads to a swelling of the silicone. Thus, the drying of step (ii) is typically drying of the swollen silicone obtained after the application of the solution in step (i). The drying may be done at ambient temperature (i.e. passive drying) or alternatively an active drying step may be performed. In some embodiments, the drying may be done at about 20° C. (i.e. at ambient temperature), e.g. for 6 to 24 hours, optionally for about 12 hours. The solvent(s) used as the swelling agent and/or to deliver the compound of formula (I) into the silicone evaporates off during the drying step; the drying time and conditions will vary depending on the solvent(s) used. Modestly elevated temperatures may be used to accelerate drying times. Embodiments of other aspects of the invention described herein apply, mutatis mutandis, to this aspect of the invention. For example, preferred compounds of formula (I).

Preferably this swell-dry process is reversible and thus does not cause a material change in the volume and shape of the silicone substrate. Without wishing to be bound by theory, it is believed that this process results in only the external portion of the silicone substrate being impregnated with a compound of formula (I) and this reduces the amount of compound required to provide a useful formulation of the invention (or device made up of or comprising said formulation). Thus preferably less than 95%, 90%, 70%, 60%, 50%, 40%, 30%, 20% or 10% of the total thickness of the silicone substrate may be impregnated with a compound of formula (I) according to this technique. Alternatively, the swell-dry method may be used to impregnate the entire silicone substrate with a compound of formula (I).

It is believed that during the swelling process a concentration gradient may be present with the outer layer of the silicone being saturated and the inner layers (furthest from the surface) being only partially saturated (or not saturated). By stopping the swelling process before total saturation (or impregnation) of the whole silicone substrate is reached, a formulation may be obtained in which the surface/outer layers contain a desired concentration of compound. The inner layers of the silicone may thus contain less compound than at the surface/outer layers (or contain no compound). Put another way, and again without being bound by theory, by controlling the swelling time the degree to which (or depth to which) the swelling agent (and thus the compound) penetrates the silicone can be controlled. Thus, by controlling the swelling time, impregnation of silicone can be controlled to achieve, if desired, impregnation of only the surface/outer layers of silicone. The thickness of the impregnated portion (or layer) could then determine the leaching rate of the compound and the time-to-depletion of the compound.

Any suitable solvent for the solution of a compound of formula (I) may be used. In preferred embodiments two miscible solvents are selected, a first solvent to dissolve the compound of formula (I) and a miscible second solvent which is particularly suitable for swelling (i.e. penetrating) the silicone substrate. Suitable first solvents include alcohols such as ethanol and 2-propanol, suitable second solvents include chloroform and pentane. In some embodiments, the solvent applied to the silicone is an ethanol/chloroform mix (e.g. a 2:1 ethanol:chloroform mix) or a propanol/pentane mix (e.g. a 2:1 to 1:2 propanol:pentane mix). The compound of formula (I) may firstly be dissolved in one solvent and then the solution mixed with the second solvent to provide the solution to be applied, also known as the swelling agent.

Typically a solution of a compound of formula (I) would be applied to the silicone such that a therapeutically effective (antimicrobially effective) amount of the compound is impregnated into the silicone. Preferably a therapeutically effective amount both when leached and as anti-colonizing agent. In some cases, a solution of a compound of formula (I) may be applied to the silicone such that there is at least 0.05 mg, at least 0.1 mg, at least 0.5 mg or at least 1 mg of the compound impregnated per cm² of the silicone.

In some embodiments, the solution of a compound of formula (I) used to swell the silicone (the swelling agent) has a concentration of compound of formula (I) of 0.25%-10%, 0.5%-10%, 1%-10%, 2%-10% or 5%-10%, 0.25%-5%, 0.5%-5%, 1%-5%, 2%-5%, 0.25%-3%, 0.5%-3%, 1%-3% (e.g. weight/volume %). In some embodiments, the solution of a compound of formula (I) used to swell the silicone has a concentration of compound of formula (I) of up to 1%, up to 2%, up to 3%, or up to 5% (e.g. weight/volume %). In some embodiments, the solution of a compound of formula (I) used to swell the silicone has a concentration of compound of formula (I) of at least 0.5%, at least 1%, at least 2% or at least 3% (e.g. weight/volume.

In some embodiments of methods of producing silicone impregnated with a compound of formula (I) that comprise applying a solution of a compound of formula (I) to the silicone substrate to swell the silicone, the solution may be applied for between 10 seconds and 6 hours, between 10 seconds and 3 hours, between 30 seconds and 180 minutes, between 30 seconds and 120 minutes, between 1 minute and 2 hours or between 2 minutes and 2 hours.

The length of time of the application (swelling time) may be chosen based on the nature (e.g. thickness, porosity) of the silicone to be impregnated and/or the depth to which impregnation of the silicone is desired.

It will be appreciated that the formulations of the invention do not comprise a silicone substrate, material or device with a compound of formula (I) simply attached to the surface thereof, rather the compound is dispersed through or blended in some or all of the silicone, thus providing a homogenous product or an (outer) layer which contains the dispersed/blended compound. Thus the formulations of the invention comprise a silicone substrate impregnated with a compound of formula (I). This property provides for controlled release of the compound when the silicone material is placed in situ, e.g. in an indwelling device or wound dressing.

In a further aspect, the present invention provides a silicone substrate which comprises a compound of formula (I) and has been produced in accordance with any of the methods described above, in particular a method where the silicone is cured in the presence of a compound of formula (I) or in which the compound of formula (I) has been introduced by a swell-dry method as described above.

The formulation of the invention may be used as an antimicrobial article. A further aspect of the present invention therefore provides, an antimicrobial article comprising the formulation of the invention. Embodiments of other aspects of the invention described herein apply, mutatis mutandis, to this aspect of the invention.

In an embodiment, the antimicrobial article is a medical device such as a catheter or an implant. Suitable implants include orthopedic implants such as hip and knee implants, as well as dental implants, pins, stents and breast implants. The formulations may also be used in or as dressings or transdermal patches, for example as an antimicrobial layer in a multi-layer dressing.

In another embodiment, the antimicrobial article is a dressing comprising a layer made from the formulation of the invention. Preferably, the dressing is a wound dressing. In an embodiment, the wound dressing consists of a layer (or mesh or sheet or the like) of silicone compounded with a compound of formula (I). In another embodiment, the dressing is a multi-layer dressing. For example, in some embodiments, a multi-layer wound dressing may comprise a layer (or mesh or sheet or the like) of silicone compounded with a compound of formula (I) and one or more additional wound dressing components, for example an additional absorbent layer and/or a secondary layer (or outer layer or cover layer or backing layer) that, for example, may be act to secure the wound dressing to the skin. The multi-layer wound dressing may comprise other wound dressing components, e.g. gauzes, bandages and/or secondary dressings.

The wound may be a partial thickness wound or a full thickness wound. The wound may be a skin tear, abrasion, laceration (cut) or burn (e.g. first or second degree burn). The wound may be an incisional wound. The wound may be an excisional wound. The wound may be a surgical wound.

The wounds may be acute or chronic. Acute wounds are wounds that proceed orderly through the three recognised stages of the healing process (i.e. the inflammatory stage, the proliferative stage and the remodelling phase) without a protracted time course. Chronic wounds, however, are those wounds that do not complete the ordered sequence of biochemical events because the wound has stalled in one of the healing stages. Viewed alternatively a chronic wound is a wound that has not healed within at least 40 days, preferably at least 50 days, more preferably at least 60 days, most preferably at least 70 days. In some embodiments chronic wounds are preferred.

The wound to be treated may be a breach in, or denudement of, the tissue for instance caused by surgical incision or trauma, e.g., mechanical, thermal, electrical, chemical or radiation trauma; a spontaneously forming lesion such as a skin ulcer (e.g. a venous, diabetic or pressure ulcer); a blister (e.g. a friction or thermal blister or a blister caused by pathogen infection such as chicken pox); an anal fissure or a mouth ulcer.

In an embodiment, the antimicrobial article is a contact lens.

In a further embodiment the antimicrobial article is a prosthetic liner.

In a further embodiment the antimicrobial article is a (trans)dermal patch, e.g. an adhesive (trans)dermal patch.

A further aspect of the invention provides an adhesive, in particular a Pressure Sensitive Adhesive, for example for securing a dressing or a medical device to a patient's skin comprising the formulation of the invention. Embodiments of other aspects of the invention described herein apply, mutatis mutandis, to this aspect of the invention. Flowable silicone substrates of the invention may be used to provide a layer or coating of silicone with a compound of formula (I) dispersed therein.

The formulation of the invention may be added as a coating to a medical device. Thus a further aspect of the present invention is a medical device coated, which includes partially coated, with a formulation of the invention as defined herein. In a yet further aspect of the invention is provided a formulation of the invention as defined herein which has been applied to a medical device. Devices include sutures, surgical fasteners, catheters, lines etc. and implants including orthopedic implants such as hip and knee implants, as well as dental implants, pins and stents.

The device to be coated may be dipped (perhaps several times, e.g. 3-10 times), in either a curable silicone elastomer composition comprising a compound of Formula (I) which may then be cured on the device under suitable curing conditions. Such dipping methods are particularly suited to devices such as sutures. Alternatively, a formulation of the invention may be applied to the medical device, e.g. an implant, by painting onto the surface of the device, e.g. by spray painting. Medical devices incorporating a formulation of the invention may also be produced by 3-D printing.

Suitable sutures to which the formulation of the invention may be applied include absorbable, optionally braided sutures. Such sutures may be made of nylon and include Surgilon and Nurolan sutures. It may be preferable to use a suture which is free of or has reduced levels of any coating other than the formulation of the invention. For example, sutures may be first treated to remove silicon coatings.

In another aspect, the invention provides a method of producing a medical device of the invention, said method comprising (i) providing a curable silicone elastomer composition comprising a compound of formula (I); (ii) applying said composition to the medical device (e.g. by dipping the device into said formulation or painting said formulation onto the device) and (iii) curing the composition on the device. Embodiments of other aspects of the invention described herein apply, mutatis mutandis, to this aspect of the invention.

A further aspect provides a formulation comprising a silicone substrate which comprises a compound of formula (I), or an antimicrobial article or adhesive comprising such a formulation, wherein the formulation is produced by a method of the invention. Embodiments of other aspects of the invention described herein apply, mutatis mutandis, to this aspect of the invention.

A further aspect of the present invention provides the formulation, antimicrobial article or adhesive of the invention for use in therapy.

“Therapy” includes treatment and prophylaxis, i.e. it includes both treatment and preventative uses.

In some embodiments, the invention provides a formulation, antimicrobial article or adhesive of the invention for use in the treatment or prevention of an infection of a subject. In some preferred embodiments, the infection or potential infection is a surgical site infection or a wound infection. In other preferred embodiments, the infection or potential infection is one associated with an implant (as discussed above) and includes biofilm formation on or around the device.

Preferably, the infection is a bacterial infection, for example an infection by Gram-positive bacteria (e.g. bacteria of the genus Staphylococcus or Streptococcus). In some embodiments, the infection is a Staphylococcus aureus infection. In some embodiments, the infection is a Staphylococcus epidermidis, E. coli or P. aeruginosa infection.

A further aspect of the present invention provides a formulation, antimicrobial article or adhesive of the present invention for use in inhibiting bacterial growth in a subject. Embodiments of other aspects of the invention described herein apply, mutatis mutandis, to this aspect of the invention.

A further aspect of the invention provides a formulation, antimicrobial article or adhesive of the present invention for use in therapy, preferably for use in the treatment or prevention of an infection of a subject. Embodiments of other aspects of the invention described herein apply, mutatis mutandis, to this aspect of the invention.

Alternatively viewed, the present invention provides a method of treating or preventing an infection which method comprises applying (or administering) to a subject in need thereof a therapeutically effective amount of formulation, antimicrobial article or adhesive of the present invention. Embodiments of other aspects of the invention described herein apply, mutatis mutandis, to this aspect of the invention.

A therapeutically effective amount will be determined based on the clinical assessment and the MIC values against target bacteria for the chosen compound of formula (I).

A further aspect of the invention provides a compound of formula (I) for use in therapy, preferably for use in the treatment or prevention of an infection of a subject, wherein said compound is administered to (or applied to) a subject as a formulation comprising a silicone compounded with said compound, or in the form of an antimicrobial article or adhesive of the invention. Embodiments of other aspects of the invention described herein apply, mutatis mutandis, to this aspect of the invention.

The term “subject” or “patient” as used herein includes any mammal, for example humans and any livestock, domestic or laboratory animal. Specific examples include mice, rats, pigs, cats, dogs, sheep, rabbits, cows and monkey. Preferably, however, the subject or patient is a human subject. Thus, subjects or patients treated in accordance with the present invention will preferably be humans.

In some embodiments, subjects or patients are those having an infection, or those suspected of having an infection, or those at risk of having (or contracting) an infection. At risk patients are a preferred patient group and include those who require a medical implant or who must undergo surgery or need to have a surgical or other wound closed. For these patients, a medical device, dressing or adhesive in accordance with the invention can be selected which incorporates a formulation of the invention. Such devices release the antimicrobial compound of Formula (I) to encourage an infection-free local environment in the patient's body around the device, and the presence of the compound of Formula (I) on/within the device inhibits colonization of the device itself by bacteria.

The invention also provides kits comprising one or more of the formulation, antimicrobial article or adhesive of the invention. Preferably said kits are for use in the therapeutic methods and uses described herein. Preferably said kits comprise instructions for use of the kit components. Preferably said kits are for treating or preventing infection, e.g. as described elsewhere herein, and optionally comprise instructions for use of the kit components to treat such infections.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

The invention will now be further described with reference to the following non-limiting Examples and Figures, in which:

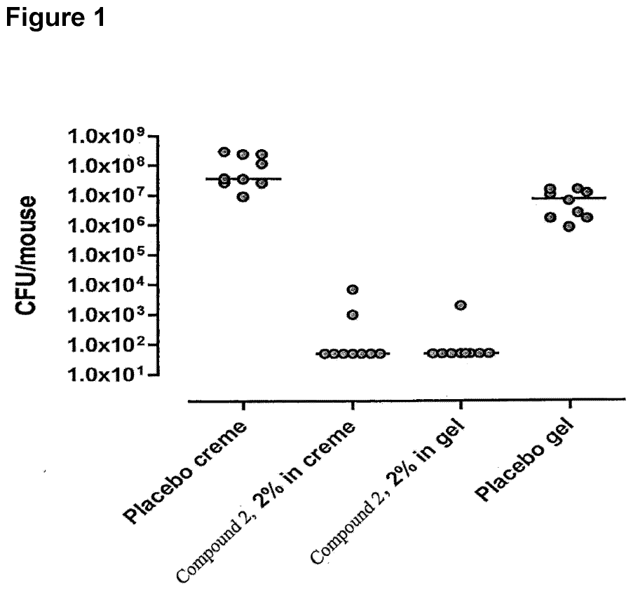

FIG. 1 is a graph showing the effect of one day topical treatment using compound 2 against Staphylococcus aureus FDA486 in a murine skin infection model. The number of colony forming units (CFU) are shown on the Y-axis and the type of topical treatment applied to the mice is shown on the X-axis. Compound 2 is also referred to herein as AMC-109.

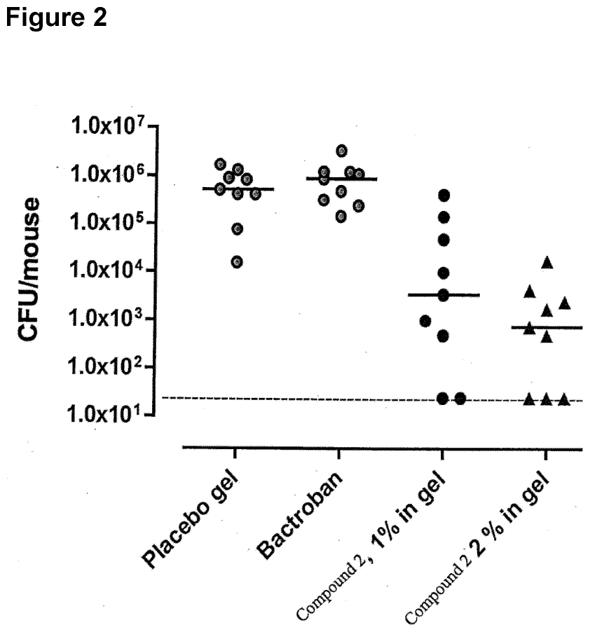

FIG. 2 is a graph showing the effect of one day topical treatment using compound 2 against Streptococcus pyogenes in a murine skin infection model. The number of colony forming units (CFU) are shown on the Y-axis and the type of topical treatment applied to the mice is shown on the X-axis. Compound 2 is also referred to herein as AMC-109.

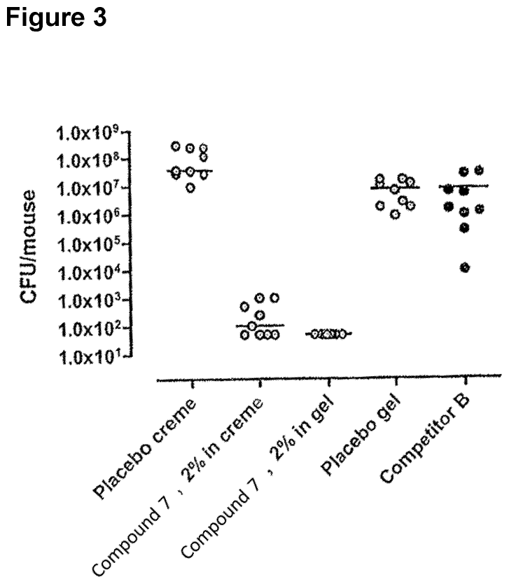

FIG. 3 is a graph showing the effect of one day topical treatment against S. aureus FDA486 in a murine skin infection model. Each mouse was treated at 9 am, 12 noon and 3 pm. The skin biopsy was collected at 6 pm. The median value is shown.

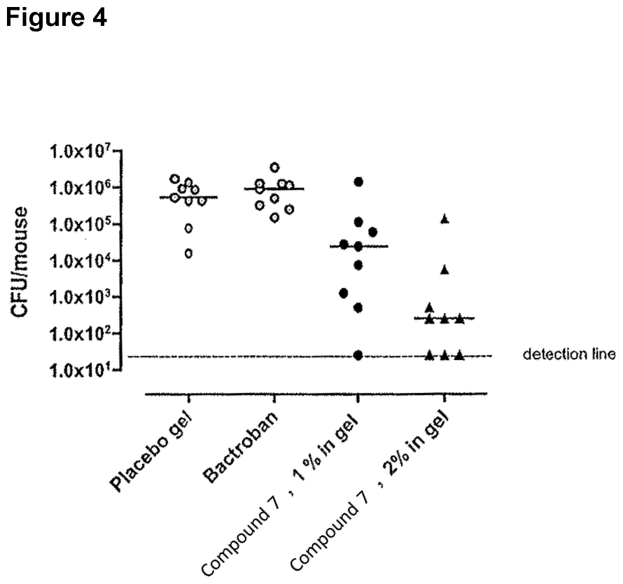

FIG. 4 is a graph showing the effect of one day topical treatment against Streptococcus pyogenes CS301 in a murine skin infection model. Each mouse was treated at 7 am, 10 am and 1 pm. The skin biopsy was collected at 4 pm. The median value is shown.

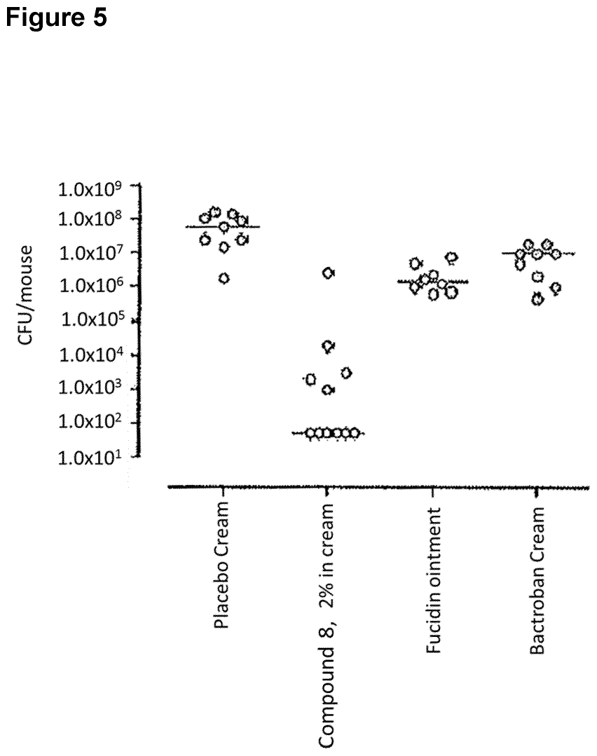

FIG. 5 is a graph showing the effect of one day topical treatment against S. aureus FDA486 in a murine skin infection model. Each mouse was treated at 9 am, 12 noon and 3 pm. The skin biopsy was collected at 6 pm. The median value is shown.

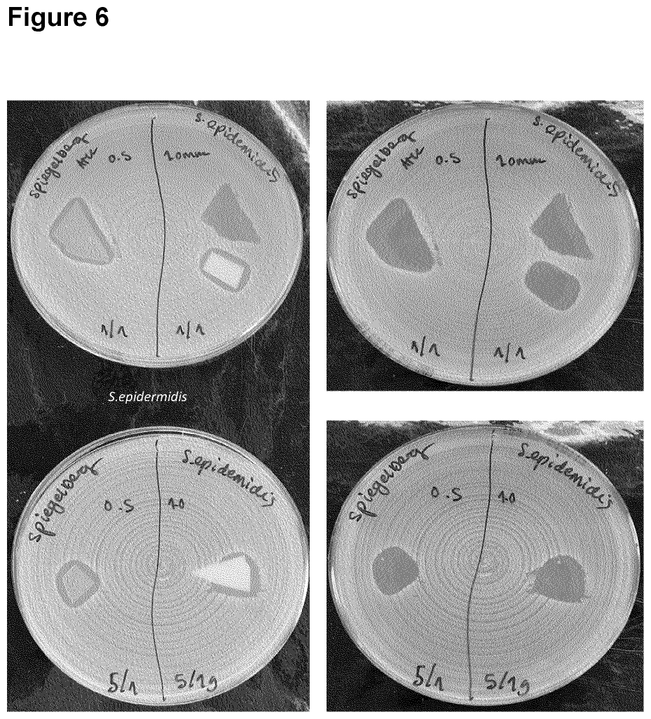

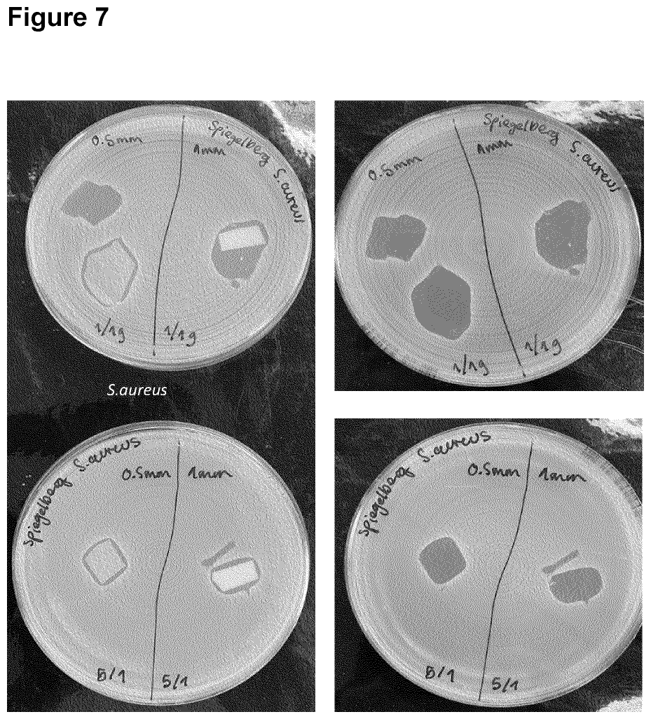

FIGS. 6 and 7 are collections of photographs showing the effect of AMC-109 compounded silicone on the growth of Staphylococcus epidermidis (FIG. 6) and Staphylococcus aureus (FIG. 7). Panel A (left) shows zones of inhibition around silicone pieces. Panel B (right) shows zones of inhibition after removal of the silicone pieces.

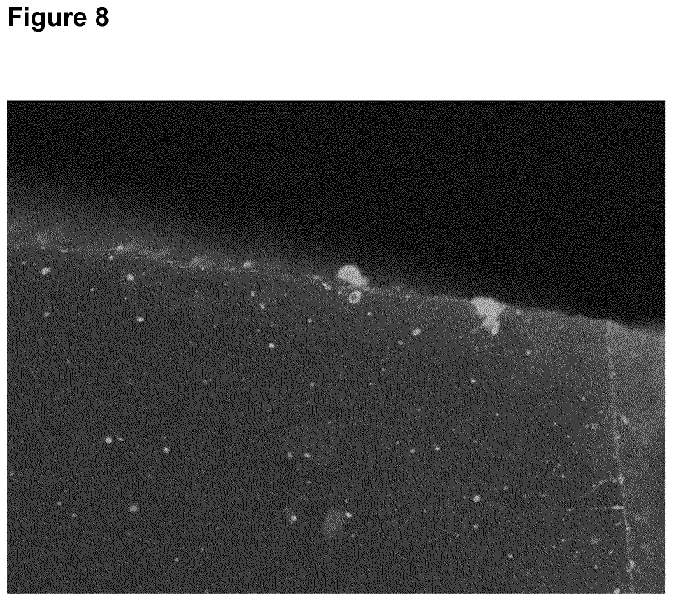

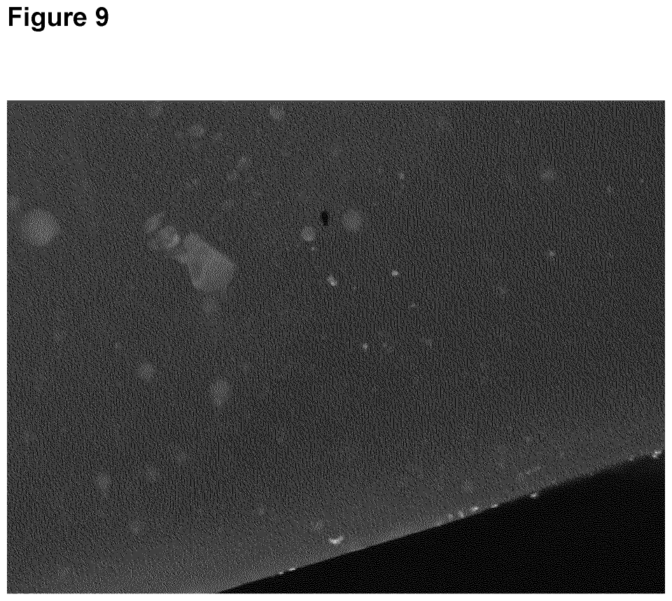

FIGS. 8 and 9 are fluorescence micrographs of sample 2/19 (left) after 16 h exposure to Staphylococcus aureus (FIG. 8) and Staphylococcus epidermidis (FIG. 9).

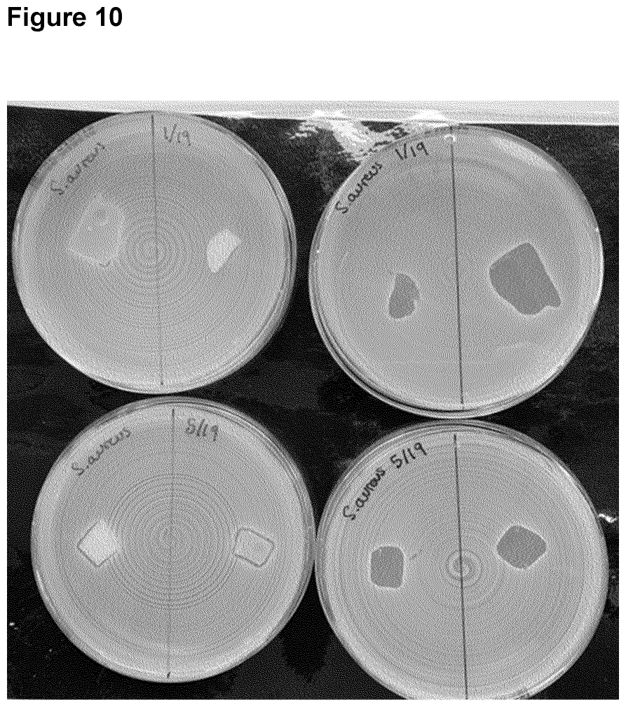

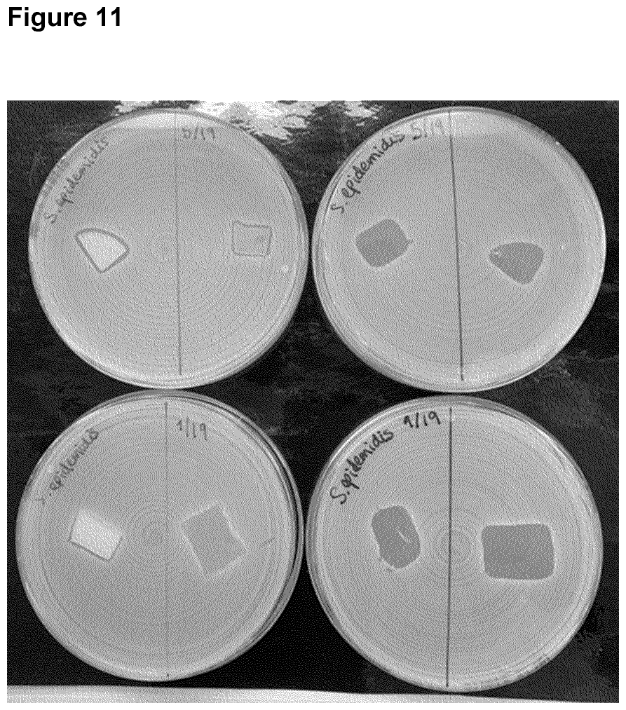

FIGS. 10 and 11 are collections of photographs showing the effect of AMC-109 compounded silicone on the growth of Staphylococcus aureus (FIG. 10) and Staphylococcus epidermidis (FIG. 11) after second use.

### EXAMPLE 1

Peptide Synthesis

Chemicals

Protected amino acids Boc-Trp-OH, Boc-Arg-OH, Boc-4-phenyl-Phe and Ac-Arg-OH were purchased from Bachem AG while Boc-4-iodophenylalanine, Boc-3,3-diphenylalanine and Boc-(9-anthryl)alanine were purchased from Aldrich. Benzylamine, 2-phenylethylamine, 3-phenylpropylamine, (R)-2-phenylpropylamine, (S)-2-phenylpropylamine, N,N-methylbenzylamine, N,N-ethylbenzylamine and N,N-dibenzylamine making up the C-terminal of the peptide were purchased from Fluka except N-ethylbenzylamine which was purchased from Acros. Diisopropylethylamine (DIPEA), 1-hydroxybenzotriazole (1-HOBt), chlorotripyrrolidinophosphonium hexafluorophosphate (PyCloP) and 0-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

Preparation of Amino Acids

Preparation of Boc-2,5,7-tri-tert-butyltryptophan-OH: A mixture of H2N-Trp-OH (1.8 g, 8.8 mmol), t-BuOH (4.7 g, 63.4 mmol) in trifluoroacetic acid (19 mL) is stirred at 70° C. for 3 hours. The volume of the resulting mid-brown translucent solution is reduced on a rotary evaporator at room temperature for 30 min and then triturated by means of adding 60 mL of 7% (by weight) NaHCO3 drop-wise. The gray/white granular solid obtained is then recovered by vacuum filtration and dried in vacuo at room temperature for 24 hours. The product is isolated by crystallization from a near boiling mixture of 40% ethanol in water. Volumes typically are approximately 20 mL per gram of crude product.

A first crystallization from crude produces isolated product of 80-83% purity (HPLC) with respect to all other substances in the sample and approximately 94-95% purity with respect to the known TBT analogues. Yields at this stage are in the range 60-65%.

Benzylation of Boc-4-iodophenylalanine. Boc-4-iodophenylalanine (1 equivalent) was dissolved in 90% methanol in water and neutralized by addition of cesium carbonate until a weak alkaline pH (determined by litmus paper). The solvent was removed by rotary evaporation, and remaining water in the cesium salt of Boc-4-iodophenylalanine was further reduced by repeated azeotropic distillation with toluene. The resulting dry salt was dissolved in dimethylformamide (DMF), benzylbromide (1.2 equivalents) was added and the resulting mixture was stirred for 6-8 h. At the end of the reaction DMF was removed under reduced pressure and an oil containing the title compound is formed. This oil was dissolved in ethyl acetate and the resulting solution was washed with equal volumes of citric acid solution (three times), sodium bicarbonate solution and brine. The title compound was isolated as a pale yellow oil in 85% yield by flash chromatography using dichloromethane:ethyl acetate (95:5) as eluent. Crystalline benzyl Boc-4-iodophenylalanine could be obtained by recrystallisation from n-heptane.

General procedure for Suzuki couplings: Benzyl Boc-4-iodophenylalanine (1 equivalent), arylboronic acid (1.5 equivalents), sodium carbonate (2 equivalents), palladium acetate (0.05 equivalent) and tri ortho-tolylphosphine (0.1 equivalent) was added to a degassed mixture of dimethoxyethane (6 ml/mmol benzyl Boc-4-iodophenylalanine) and water (1 ml/mmol benzyl Boc-4-iodophenylalanine). The reaction mixture was kept under argon and heated to 80° C. for 4-6 h. After cooling to room temperature the mixture is filtered through a short pad of silicagel and sodium carbonate. The filter cake was further washed with ethyl acetate. The filtrates were combined and the solvents were removed under reduced pressure. The products were isolated by flash chromatography using mixtures of ethyl acetate and n-hexane as eluent.

Preparation of Boc-Bip(n-Bu)-OBn: The title compound was prepared in 53% yield from 4-n-butylphenylboronic acid using the general procedure for Suzuki couplings. Boc-Bip(n-Bu)-OBn was isolated using an 80:20 ethyl acetate:n-hexane eluent.

Preparation of Boc-Bip(t-Bu)-OBn: The title compound was prepared in 79% yield from 4-t-butylphenylboronic acid using the general procedure for Suzuki couplings. Boc-Bip(t-Bu)-OBn was isolated using an 80:20 ethyl acetate:n-hexane eluent.

Preparation of Boc-Bip(4-Ph)-OBn: The title compound was prepared in 61% yield from 4-biphenylboronic acid using the general procedure for Suzuki couplings. Boc-Bip(4-Ph)-OBn was isolated by recrystallisation of the crude product from n-heptane.

Preparation of Boc-Bip(4-(2-Naphtyl))-OBn: The title compound was prepared in 68% yield from 2-naphthylboronic acid using the general procedure for Suzuki couplings. Boc-Bip(4-(2-Naphtyl))-OBn was isolated by recrystallisation of the crude product from n-heptane.

Preparation of Boc-Bip(4-(1-Naphtyl))-OBn: The title compound was prepared from 2-naphthylboronic acid using the general procedure for Suzuki couplings. Boc-Bip(4-(1-Naphtyl))-OBn was isolated by recrystallisation of the crude product from n-heptane.

General procedure for deesterification of benzyl esters: The Benzyl ester is dissolved in DMF and hydrogenated for 2 days at ambient pressure using 10% Pd on carbon as catalyst. At the end of the reaction the catalyst is removed by filtration and the solvent is removed under reduced pressure. The free acids are isolated by recrystallisation from diethyl ether.

Preparation of Boc-Bip(4-n-Bu)-OH: The title compound was prepared in 61% yield from Boc-Bip(n-Bu)-OBn using the general procedure for deesterification.

Preparation of Boc-Bip(4-t-Bu)-OH: The title compound was prepared in 65% yield from Boc-Bip(t-Bu)-OBn using the general procedure for deesterification.

Preparation of Boc-Bip(4-Ph)-OH: The title compound was prepared in 61% yield from Boc-Bip(4-ph)-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

General procedure for Solution phase peptide synthesis using HBTU. The peptides were prepared in solution by stepwise amino acid coupling using Boc protecting strategy according to the following general procedure. The C-terminal peptide part with a free amino group (1 eq) and the Boc protected amino acid (1.05 eq) and 1-hydroxybenzotriazole (1-HOBt) (1.8 eq) were dissolved in DMF (2-4 ml/mmol amino component) before addition of diisopropylethylamine (DIPEA) (4.8 eq). The mixture was cooled on ice and O-(benzotriazol-1-yl)-N,N,N′,N′ tetramethyluronium hexafluorophosphate (HBTU) (1.2 eq) was added. The reaction mixture was shaken at ambient temperature for 1-2 h. The reaction mixture was diluted by ethyl acetate and washed with citric acid, sodium bicarbonate and brine. The solvent was removed under vacuum and the Boc protecting group of the resulting peptide was deprotected in the dark using 95% TFA or acetylchloride in anhydrous methanol.

Solution phase amide formation using PyCloP. Synthesis of Boc-Arg-N(CH₂Ph)₂. A solution of Boc-Arg-OH (1 eq), NH(CH₂Ph)₂ (1.1 eq) and PyCloP (1 eq) in dry DCM (filtered through alumina) (2 ml) and DMF (1 ml). The solution was cooled on ice and DIPEA (2 eq) was added under stirring. The solution was stirred for 1 h at room temperature. The reaction mixture was evaporated, and redissolved in ethyl acetate and washed with citric acid, sodium bicarbonate and brine. The solvent was removed under vacuum and the Boc protecting group of the resulting peptide was deprotected in the dark using 95% TFA.

Peptide purification and analysis. The peptides were purified using reversed phase HPLC on a Delta-Pak (Waters) 018 column (100 Å, 15 μm, 25×100 mm) with a mixture of water and acetonitrile (both containing 0.1% TFA) as eluent. The peptides were analyzed by RP-HPLC using an analytical Delta-Pak (Waters) 018 column (100 Å, 5 μm, 3.9×150 mm) and positive ion electrospray mass spectrometry on a VG Quattro quadrupole mass spectrometer (VG Instruments Inc., Altringham, UK).

### EXAMPLE 2

In Vitro Activities of Peptides Defined Herein

Materials and Methods

Antimicrobials

Vials of pre-weighed Compound 1 and Compound 2 were supplied by Lytix Biopharma AS.

General compound formula: AA₁-AA₂-AA₁-XY

| | AA₁ | AA₂ | XY |
|---|---|---|---|
| Compound 1 | Arg | Phe(4-(2-Naphtyl)) | NHCH₂CH₂Ph |
| Compound 2 | Arg | 2,5,7-tri-tert- | NHCH₂CH₂Ph |
| | | butyltryptophan | |

Bacterial Isolates

Bacterial isolates used in this study were from various sources worldwide stored at GR Micro Ltd. and maintained, with minimal sub-culture, deep frozen at −70° C. as a dense suspension in a high protein matrix of undiluted horse serum. The species used and their characteristics are listed in Table 1. These included 54 Gram-positive bacteria, 33 Gram-negative bacteria and 10 fungi.

Determination of Minimum Inhibitory Concentration (MIC)

MICs were determined using the following microbroth dilution methods for antimicrobial susceptibility testing published by the Clinical and Laboratory Standards Institute (CLSI, formerly NCCLS):

M7-A6 Vol. 23 No. 2 Jan. 2003 Methods for Dilution Antimicrobial Susceptibility Tests for Bacteria that Grow Aerobically; Approved Standard—Sixth Edition. M100-S15 Vol. 25 No 1. January 2005 Performance Standards for Antimicrobial Susceptibility Testing; Fifteenth Informational Supplement. M11-A6 Vol. 24 No. 2 Methods for Antimicrobial Susceptibility Testing of Anaerobic Bacteria; Approved Standard—Sixth Edition. M27-A2 Vol. 22 No. 15 Reference Method for Broth Dilution Antifungal Susceptibility Testing of Yeasts; Approved Standard—Second Edition. M38-A Vol. 22 No. 16 Reference Method for Broth Dilution Antifungal Susceptibility Testing of Filamentous Fungi; Approved Standard.

MIC estimations were performed using wet plates, containing the antibacterials or antifungals, prepared at GR Micro Ltd.

Cation-adjusted Mueller-Hinton broth (Oxoid Ltd., Basingstoke, UK and Trek Diagnostic Systems Ltd., East Grinstead, UK) (supplemented with 5% laked horse blood for Streptococcus spp., Corynebacterium jeikeium and Listeria monocytogenes) was used for aerobic bacteria, with an initial inoculum of approximately 10⁵ colony-forming units (CFU)/m L.

Haemophilus test medium (Mueller-Hinton broth containing 0.5% yeast extract and Haemophilus test medium supplement which contains 15 mg/L of each of haematin and NAD, all obtained from Oxoid Ltd., Basingstoke, UK) was used for the Haemophilus influenzae and inoculated with approximately 10⁵ CFU/mL.

Supplemented Brucella broth (SBB) was used for the anaerobic strains with an inoculum of approximately 10⁶ CFU/mL. SBB is a broth consisting of 1% peptone, 0.5% ‘Lab-lemco’, 1% glucose and 0.5% sodium chloride supplemented with 5 μg/L haemin and 1 μg/L vitamin K (both obtained from Sigma Aldrich Ltd.) Yeast and filamentous fungal MIC were performed in MOPS buffered RPMI 1640 medium (MOPS buffer obtained from Sigma Aldrich Ltd., RPMI 1640 obtained from Invitrogen Ltd, Paisley, Scotland). The yeast inocula were in the range 7.5×10²-4×10³ CFU/mL and the filamentous fungi approximately 8×10³-1×10⁵ CFU/mL

Following normal practice all the plates containing Mueller-Hinton broth were prepared in advance, frozen at −70° C. on the day of preparation and defrosted on the day of use. Fungal, Haemophilus and anaerobic MIC determinations were all performed in plates prepared on the same day.

To evaluate whether freezing affected the activity of the peptides some MIC determinations were repeated using plates containing freshly-prepared Mueller-Hinton broth.

Control Strains

The following control (reference) strains were included in the panel of strains tested

| | |
|---|---|
| Escherichia coli | ATCC 25922 |
| Staphylococcus aureus | ATCC 29213 |
| Enterococcus faecalis | ATCC 29212 |
| Streptococcus pneumoniae | ATCC 49619 |
| Pseudomonas aeruginosa | ATCC 27853 |
| Candida krusei | ATCC 6258 |

The control strains below were extra to the test strain panel and were included where appropriate, to check that the comparators were within range.

| | |
|---|---|
| Haemophilus influenzae | ATCC 49247 |
| Candida parapsilosis | ATCC 22019 |
| Bacteroides fragilis | ATCC 25285 |
| Eggerthella lenta | ATCC 43055 |

Results

The results are shown in Table 1 as a single line listing. Repeat control strain results are shown in Table 2. It can be seen that the control strain results were highly reproducible including data from plates that contained Mueller Hinton broth either stored frozen or used fresh. Freezing plates also had no effect on the MIC for other bacterial strains.

The MIC data obtained is very encouraging and indicates that the peptides have quite a broad spectrum of activity.

TABLE 1
Single line list of the in vitro activity of two antimicrobial peptides and a comparator
against a panel of Gram-positive bacteria, Gram-negative bacteria and fungi.

| | Compound | Compound |
|---|---|---|
| Species and properties | 1 | 2 |
| Candida albicans ATCC90028 - reference strain | 2 | 8 |
| Candida albicans ATCC24433 - reference strain | 2 | 8 |
| Candida tropicalis ATCC750 - reference strain | 2 | 4 |
| Candida parapsilosis ATCC90018 - reference strain | 4 | 16 |
| Candida (Issatchenkia) krusei ATCC6258 - reference strain | 4 | 4 |
| Aspergillus niger - G.R. Micro collection | 4 | 8 |
| Trichophyton mentagrophytes - G.R. Micro collection | 16 | 8 |
| Trichophyton interdigitale - G.R. Micro collection | 8 | 8 |
| Microsporum canis - G.R. Micro collection | 8 | 8 |
| Cryptococcus neoformans - G.R. Micro collection | 4 | 4 |
| Escherichia coli ATCC25922 - antibiotic-susceptible type strain | 32 | 8 |
| Escherichia coli ATCC32518 - β-lactamase positive type strain | 32 | 8 |
| Escherichia coli - multi-drug resistant clinical isolate | 32 | 8 |
| Klebsiella aerogenes NCTC11228 - antibiotic-susceptible type strain | 32 | 16 |
| Klebsiella aerogenes - multi-drug resistant clinical isolate | 64 | 16 |
| Enterobacter sp - antibiotic-susceptible clinical isolate | 32 | 4 |
| Enterobacter sp - multi-drug resistant clinical isolate | 64 | 16 |
| Pseudomonas aeruginosa ATCC27853 - antibiotic-susceptible type | 16 | 8 |
| Pseudomonas aeruginosa - multi-drug resistant clinical isolate | 32 | 4 |
| Stenotrophomonas maltophilia - antibiotic-susceptible clinical isolate | 64 | 8 |
| Salmonella sp - antibiotic-susceptible clinical isolate | 16 | 8 |
| Salmonella sp - multi-drug resistant clinical isolate | 16 | 8 |
| Shigella sp - antibiotic-susceptible clinical isolate | 32 | 8 |
| Morganella morganii - multi-drug resistant clinical isolate | ≥128 | 16 |
| Haemophilus influenzae - β- lactamase negative clinical isolate | ≥128 | 8 |
| Haemophilus influenzae - β -lactamase positive clinical isolate | ≥128 | 8 |
| Haemophilus influenzae β- lactamase negative ampicillin-resistant | ≥128 | 8 |
| Moraxella catarrhalis - β -lactamase positive clinical isolate | 4 | 4 |
| Moraxella catarrhalis - reduced fluoroquinolone susceptibility clinical | 8 | 8 |
| Acinetobacter baumanii - antibiotic-susceptible clinical isolate | 64 | 16 |
| Staphylococcus aureus ATCC 29213 - antibiotic-susceptible control | 4 | 2 |
| Staphylococcus aureus ATCC 25923 - antibiotic-susceptible control | 4 | 4 |
| Staphylococcus aureus ATCC 43300 - methicillin-resistant control | 4 | 2 |
| strain | | |
| Staphylococcus aureus - methicillin-resistant clinical isolate | 4 | 4 |
| Staphylococcus aureus - multi-drug-resistant clinical isolate | 8 | 4 |
| Staphylococcus aureus - teicoplanin-intermediate clinical isolate | 4 | 4 |
| Staphylococcus epidermidis antibiotic susceptible clinical isolate | 16 | 8 |
| Staphylococcus epidermidis methicillin-resistant clinical isolate | 2 | 4 |
| Staphylococcus haemolyticus - antibiotic susceptible clinical isolate | 4 | 4 |
| Staphylococcus saprophyticus - antibiotic susceptible clinical isolate | 1 | 1 |
| Enterococcus faecalis - ATCC 29212 antibiotic-susceptible control | 4 | 4 |
| Enterococcus faecalis vancomycin-susceptible clinical isolate | 8 | 8 |
| Enterococcus faecalis vancomycin-resistant (VanA) clinical isolate | 16 | 8 |
| Enterococcus faecalis vancomycin-resistant (VanB) clinical isolate | 16 | 16 |
| Enterococcus faecalis high-level gentamicin-resistant clinical isolate | 16 | 8 |
| Enterococcus faecium vancomycin-susceptible clinical isolate | 8 | 8 |
| Enterococcus faecium vancomycin-resistant (VanA) clinical isolate | 16 | 8 |
| Enterococcus faecium vancomycin-resistant (VanB) clinical isolate | 8 | 4 |
| Enterococcus gallinarum vancomycin-resistant (VanC) clinical isolate | 4 | 4 |
| Streptococcus pneumoniae - ATCC 49619 antibiotic-susceptible | 32 | 16 |
| control | | |
| Streptococcus pneumoniae - penicillin-susceptible clinical isolate | 64 | 32 |
| Streptococcus pneumoniae - penicillin-intermediate clinical isolate | 32 | 32 |
| Streptococcus pneumoniae - penicillin-resistant clinical isolate | 32 | 16 |
| Streptococcus pneumoniae - multi-drug resistant clinical isolate | 64 | 32 |
| Streptococcus pyogenes - Macrolide (MLS) resistant clinical isolate | 32 | 16 |
| Streptococcus pyogenes - Macrolide (M-type) resistance clinical | 32 | 16 |
| isolate | | |
| Corynebacterium jeikeium - antibiotic-susceptible clinical isolate | 32 | 16 |
| Corynebacterium jeikeium - multi-drug resistant clinical isolate | 32 | 8 |
| Listeria monocytogenes - antibiotic-susceptible clinical isolate | 32 | 16 |
| MU50 Staphylococcus aureus (MRSA) - VISA type strain | 4 | 4 |
| EMRSA3 Staphylococcus aureus (MRSA) - SSCmec type 1 | 4 | 4 |
| EMRSA16 Staphylococcus aureus (MRSA) - SSCmec type 2 | 4 | 4 |
| EMRSA1 Staphylococcus aureus (MRSA) - SSCmec type 3 | 8 | 8 |
| EMRSA15 Staphylococcus aureus (MRSA) - SSCmec type 4 | 4 | 4 |
| HT2001254 Staphylococcus aureus (MRSA) - PVL positive | 4 | 4 |
| Streptococcus agalactiae - antibiotic-susceptible clinical isolate | 16 | 8 |
| Streptococcus agalactiae - macrolide-resistant clinical isolate | 32 | 16 |
| Group C Streptococcus - antibiotic-susceptible clinical isolate | 32 | 16 |
| Group C Streptococcus - macrolide-resistant clinical isolate | 64 | 32 |
| Group G Streptococcus - antibiotic-susceptible clinical isolate | 32 | 8 |
| Group G Streptococcus - macrolide-resistant clinical isolate | 32 | 16 |
| Streptococcus mitis - antibiotic-susceptible clinical isolate | 64 | 16 |
| Streptococcus mitis - macrolide-resistant clinical isolate | ≥128 | 32 |
| Streptococcus constellatus - antibiotic-susceptible clinical isolate | 64 | 32 |
| Streptococcus constellatus - macrolide-resistant clinical isolate | 64 | 32 |
| Streptococcus oralis - antibiotic-susceptible clinical isolate | 64 | 32 |
| Streptococcus oralis - macrolide-resistant clinical isolate | 32 | 32 |
| Streptococcus bovis - antibiotic-susceptible clinical isolate | 64 | 32 |
| Streptococcus bovis - macrolide-resistant clinical isolate | 8 | 8 |
| Streptococcus sanguis - antibiotic-susceptible clinical isolate | 64 | 32 |
| Streptococcus sanguis - macrolide-resistant clinical isolate | 32 | 32 |
| Clostridium perfringens - antibiotic-susceptible clinical isolate | ≥128 | 32 |
| Clostridium difficile - antibiotic-susceptible clinical isolate | 32 | 16 |

TABLE 2
In vitro activity of two antimicrobial peptides and
comparators against ATCC control strains
(Including ATCC control strains extra to the test strain panel)

| Strain | | Compound | | |
|---|---|---|---|---|
| No. | Species and properties | 1 | 2 | Plate type |
| GP01 | Staphylococcus aureus | 8 | 4 | Frozen MHB |
| | ATCC 29213 antibiotic- | | | |
| | susceptible control strain | | | |
| GP01 | Staphylococcus aureus | 4 | 4 | Frozen MHB |
| | ATCC 29213 antibiotic- | | | |
| | susceptible control strain | | | |
| GP01 | Staphylococcus aureus | 4 | 2 | Fresh MHB |
| | ATCC 29213 antibiotic- | | | |
| | susceptible control strain | | | |
| GN01 | Escherichia coli | 32 | 8 | Frozen MHB |
| | ATCC 25922 antibiotic- | | | |
| | susceptible type strain | | | |
| GN01 | Escherichia coli | 32 | 8 | Frozen MHB |
| | ATCC 25922 antibiotic- | | | |
| | susceptible type strain | | | |
| GN01 | Escherichia coli | 16 | 8 | Fresh MHB |
| | ATCC 25922 antibiotic- | | | |
| | susceptible type strain | | | |
| GN10 | Pseudomonas aeruginosa | 16 | 8 | Frozen MHB |
| | ATCC 27853 antibiotic- | | | |
| | susceptible type strain | | | |
| GN10 | Pseudomonas aeruginosa | 32 | 8 | Frozen MHB |
| | ATCC 27853 antibiotic- | | | |
| | susceptible type strain | | | |
| GN10 | Pseudomonas aeruginosa | 8 | 8 | Fresh MHB |
| | ATCC 27853 antibiotic- | | | |
| | susceptible type strain | | | |
| GP11 | Enterococcus faecalis - | 8 | 8 | Frozen MHB |
| | ATCC 29212 antibiotic- | | | |
| | susceptible control strain | | | |
| GP11 | Enterococcus faecalis - | 8 | 8 | Frozen MHB |
| | ATCC 29212 antibiotic- | | | |
| | susceptible control strain | | | |
| GP11 | Enterococcus faecalis - | 4 | 4 | Fresh MHB |
| | ATCC 29212 antibiotic- | | | |
| | susceptible control strain | | | |
| | Haemophilus influenzae - | 32 | 4 | HTM |
| | ATCC 47247 | | | |
| | Candida parapsilosis | 4 | 8 | RPMI 1640 |
| | ATCC 22019 | | | |
| F05 | Candida (Issatchenkia) | 8 | 8 | RPMI 1640 |
| | krusei ATCC 6258 | | | |
| | reference strain | | | |
| F05 | Candida (Issatchenkia) | 8 | 8 | RPMI 1640 |
| | krusei ATCC | | | |
| | 6258 reference strain | | | |
| | Bacteroides fragilis - | 64 | 64 | SBB |
| | ATCC 25285 | | | |
| | Eggerthella lenta - | 16 | 32 | SBB |
| | ATCC 43055 | | | |

MHB, Mueller Hinton broth; HTM, haemophilus test medium; SBB, supplemented Brucella broth.

### EXAMPLE 3 STABILITY TOWARDS TRYPTIC DEGRADATION AND ANTIMICROBIAL ACTIVITY

Compounds of formula AA₁-AA₂-AA₁-NHCH₂CH₂Ph were tested for their trypsin resistance and antimicrobial activity.

Measurements and Calculation of Peptide Half-Life

Each peptide was dissolved in a 0.1 M NH₄HCO₃ buffer (pH 6.5) to yield a final peptide concentration of 1 mg/ml. A trypsin solution was prepared by dissolving 1 mg of trypsin in 50 ml 0.1 M NH₄HCO₃ buffer (pH 8.2). For the stability determination, 250 μl freshly made trypsin solution and 250 μl peptide solution were incubated in 2 ml of 0.1 M NH₄HCO₃ buffer (pH 8.6) at 37° C. on a rocking table. Aliquots of 0.5 ml were sampled at different time intervals, diluted with 0.5 ml water:acetonitrile (60:40 v/v) containing 1% TFA and analysed by RP-HPLC as described above. Samples without trypsin addition taken at 0 h and after 20 h at 37° C. were used as negative controls. Integration of the peak area at 254 nm for samples taken during the first 5 hours of the assay was used to generate the 11/2. Peptides that displayed no degradation during the first 24 h were classified as stable.

Antibacterial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 3
Stability of AA₁-AA₂-AA₁-NHCH₂CH₂Ph peptides towards trypsin measured
as half-life (τ_{1/2}) and antibacterial activities displayed as MIC.

| | | | | MIC^{b} (μM) | | |
|---|---|---|---|---|---|---|
| Peptide | AA₁ | AA₂ | τ_{1/2}^{a} (h) | S. aureus^{c} | MRSA^{d} | MRSE^{e} |
| Compound | Arg | Trp | 7 | 145 | 97 | 81 |
| 6^{f} | | | | | | |
| Compound | Arg | Bip(4-Ph) | Stable | 5 | 3 | 3 |
| 5 | | | | | | |
| Compound | Lys | 2,5,7-tri-tert- | Stable | | 3 | <2 |
| 4 | | butyltryptophan | | | | |
| Compound | Arg | Phe(4-(1- | 20 | | 3 | 3 |
| 3 | | Naphtyl)) | | | | |
| Compound | Arg | 2,5,7-tri-tert- | Stable | <3 | <3 | <3 |
| 2 | | butyltryptophan | | | | |
| Compound | Arg | Phe(4-(2- | Stable | 4 | <3 | <3 |
| 1 | | Naphtyl)) | | | | |

^{a}Medical Calculator from Cornell University was used to calculate the half-life.
^{b}Minimal inhibitory concentration
^{c}Staphylococcus aures strain ATCC 25923
^{d}Methicillin resistant Staphylococcus aureus ATCC 33591
^{e}Methicillin resistant Staphylococcus epidermis ATCC 27626
^{f}not within compound definition for invention

### EXAMPLE 4 IN VIVO ACTIVITY OF COMPOUND 2

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined. Results are shown in FIGS. 1 and 2, expressed as the number of colony forming units per mouse.

In experiment 1 (FIG. 1), compound 2 was applied to the murine skin as part of either a cream or a gel containing 2% (w/w) of compound 2. The same cream or gel without compound 2 was used as a negative control (placebo). It can clearly be seen that the number of CFUs was reduced when a cream or gel containing compound 2 was applied to the murine skin, compared to the negative control, indicating that compound 2 exerted an antimicrobial effect against Staphylococcus aureus. The nature of the carrier, cream or gel, had no significant effect.

In experiment 2 (FIG. 2), compound 2 was applied in two different concentrations, as either a 1% or a 2% gel. A placebo gel and a known antibacterial “bactroban” were used as controls. It can be seen that gels containing compound 2 were more effective at reducing the number of CFUs than the placebo gel or the bactroban. The gel containing 2% of compound 2 was more effective than the gel containing only 1% of compound 2.

### EXAMPLE 5

Preparation and Physical, Antimicrobial and Haemolytic Properties of Compounds of Use in the Invention

Peptide Synthesis—relevant information is also provided in Example 1.

Chemicals:

Protected amino acids Boc-Arg-OH, and Boc-4-phenyl-Phe were purchased from Bachem AG while Boc-4-iodophenylalanine was purchased from Aldrich. isopropylamine, propylamine, hexylamine, butylamine, hexadecylamine, isobutylamine, cyclohexylamine and cyclopentylamine making up the C-terminal of the peptide were purchased from Fluka. Diisopropylethylamine (DIPEA), 1-hydroxybenzotriazole (1-HOBt), chlorotripyrrolidinophosphonium hexafluorophosphate (PyCloP) and O-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

Preparation of Boc-Phe(4-4′-biphenyl)-OBn: The title compound was prepared in 61% yield from 4-biphenylboronic acid using the general procedure for Suzuki couplings. Boc-Phe(4-4′-biphenyl)-OBn was isolated by recrystallisation of the crude product from n-heptane.

Preparation of Boc-Phe(4-(2′-Naphtyl))-OBn: The title compound was prepared in 68% yield from 2-naphthylboronic acid using the general procedure for Suzuki couplings. Boc-Phe(4-(2′-Naphtyl))-OBn was isolated by recrystallisation of the crude product from n-heptane.

Preparation of Boc-Phe(4-4′-biphenyl)-OH: The title compound was prepared in 61% yield from Boc-Phe(4-4′-biphenyl)-OBn using the general procedure for deesterification.

Preparation of Boc-Phe(4-(2′-Naphtyl))-OH: The title compound was prepared in 68% yield from Boc-Phe(4-(2-Naphtyl))-OBn using the general procedure for deesterification.

General procedure for Solution phase peptide synthesis using HBTU is described in Example 1.

Solution phase amide formation using PyCloP is described in Example 1.

Peptide purification and analysis is described in Example 1.

TABLE 4
General compound formula: Arg-AA₂-Arg-X-Y

| | | | Purity |
|---|---|---|---|
| Compound | AA₂ | XY | (HPLC) |
| 7 | 2,5,7-tri-tert- | NHCH(CH₃)₂ | |
| | butyltryptophan | | |
| 8 | 2,5,7-tri-tert- | NH(CH₂)₅CH₃ | |
| | butyltryptophan | | |
| 9 | 2,5,7-tri-tert- | NH(CH₂)₃CH₃ | 87 |
| | butyltryptophan | | |
| 10 | 2,5,7-tri-tert- | NH(CH₂)₂CH₃ | 99 |
| | butyltryptophan | | |
| 11 | 2,5,7-tri-tert- | NH(CH₂)₁₅CH₃ | 80 |
| | butyltryptophan | | |
| 12 | 2,5,7-tri-tert- | NHCH₂CH(CH₃)₂ | 97 |
| | butyltryptophan | | |
| 13 | 2,5,7-tri-tert- | NHcyclohexyl | 95 |
| | butyltryptophan | | |
| 14 | 2,5,7-tri-tert- | NHcyclopentyl | 91 |
| | butyltryptophan | | |
| 15 | Phe(4-4′- | NHCH(CH₃)₂ | |
| | biphenyl) | | |
| 16 | Phe(4-4′- | NH(CH₂)₅CH₃ | |
| | biphenyl) | | |
| 17 | Phe(4-(2′- | NHCH(CH₃)₂ | |
| | Naphtyl)) | | |
| 18 | Phe(4-(2′- | NH(CH₂)₅CH₃ | |
| | Naphtyl)) | | |

Antimicrobial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 5
Antimicrobial and toxic properties of compounds of use in the invention

| | C. albicans | S. aureus | MRSA | MRSE | S. pyogenes | E. coli | P. aeruginosa | |
|---|---|---|---|---|---|---|---|---|
| Compound | (mg/L) | (mg/L) | (mg/L) | (mg/L) | (mg/L) | (mg/L) | (mg/L) | EC50 |
| 7 | 25 | <2 | <2 | <2 | <2 | 7 | 7 | 720 |
| 8 | 5 | 2 | 2 | <1 | 2 | 5 | 5 | 32 |
| 9 | 10 | 2 | 3 | <2 | 2 | | | 350 |
| 10 | 10 | 2 | 3 | <2 | 2 | | | 620 |
| 11 | >100 | 5 | 4 | 4 | 6 | >100 | >100 | 38 |
| 12 | 10 | <2 | 3 | 2 | 2 | | | 300 |
| 13 | 10 | <2 | 2 | 2 | <2 | | | 55 |
| 14 | 10 | <2 | >15 | <2 | 2 | | | 340 |

### EXAMPLE 6

In Vitro Broad Panel Screening of Selected Compounds

Materials and Methods

Antimicrobials

Vials of pre-weighed Compound 7 and Compound 8 were supplied by Lytix Biopharma AS.

General compound formula: AA₁-AA₂-AA₁-X-Y

| | AA₁ | AA₂ | XY |
|---|---|---|---|
| Compound 7 | Arg | 2,5,7-tri-tert- | NHCH(CH₃)₂ |
| | | butyltryptophan | |
| Compound 8 | Arg | 2,5,7-tri-tert- | NH(CH₂)₅CH₃ |
| | | butyltryptophan | |

Bacterial Isolates

Bacterial isolates used in this study are as described in Example 2.

Determination of Minimum Inhibitory Concentration (MIC)

MICs were determined as described in Example 2.

Results

The results are shown in Table 6 as a single line listing.

The MIC data obtained is very encouraging and indicates that the peptides have quite a broad spectrum of activity.

TABLE 6
Single line list of the in vitro activity of two antimicrobial peptides against a
panel of Gram-positive bacteria, Gram-negative bacteria and fungi.

| | Compound | Compound |
|---|---|---|
| Species and properties | 7 (mg/L) | 8 (mg/L) |
| Candida albicans ATCC90028 - reference strain | 32 | 4 |
| Candida albicans ATCC24433 - reference strain | 64 | 8 |
| Candida tropicalis ATCC750 - reference strain | 4 | 4 |
| Candida parapsilosis ATCC90018 - reference strain | 64 | 8 |
| Candida (Issatchenkia) krusei ATCC6258 - reference strain | 8 | 32 |
| Aspergillus niger - G.R. Micro collection | 32 | 4 |
| Trichophyton mentagrophytes - G.R. Micro collection | 8 | 4 |
| Trichophyton interdigitale - G.R. Micro collection | 16 | 4 |
| Microsporum canis - G.R. Micro collection | 16 | 4 |
| Cryptococcus neoformans - G.R. Micro collection | 8 | 2 |
| Escherichia coli ATCC25922 - antibiotic-susceptible type strain | 32 | 4 |
| Escherichia coli ATCC32518 - β-lactamase positive type strain | 32 | 8 |
| Escherichia coli - multi-drug resistant clinical isolate | 32 | 8 |
| Klebsiella aerogenes NCTC11228 - antibiotic-susceptible type strain | 64 | 8 |
| Klebsiella aerogenes - multi-drug resistant clinical isolate | 32 | 8 |
| Enterobacter sp - antibiotic-susceptible clinical isolate | 64 | 8 |
| Enterobacter sp - multi-drug resistant clinical isolate | ≥128 | 8 |
| Pseudomonas aeruginosa ATCC27853 - antibiotic-susceptible type strain | 32 | 8 |
| Pseudomonas aeruginosa - multi-drug resistant clinical isolate | 8 | 4 |
| Stenotrophomonas maltophilia - antibiotic-susceptible clinical isolate | 32 | 4 |
| Salmonella sp - antibiotic-susceptible clinical isolate | 16 | 8 |
| Salmonella sp - multi-drug resistant clinical isolate | 16 | 8 |
| Shigella sp - antibiotic-susceptible clinical isolate | 32 | 4 |
| Morganella morganii - multi-drug resistant clinical isolate | 32 | 8 |
| Haemophilus influenzae - β- lactamase negative clinical isolate | 32 | 16 |
| Haemophilus influenzae - β -lactamase positive clinical isolate | 16 | 4 |
| Haemophilus influenzae β- lactamase negative ampicillin-resistant clinical | 16 | 8 |
| isolate | | |
| Moraxella catarrhalis - β -lactamase positive clinical isolate | 4 | 16 |
| Moraxella catarrhalis - reduced fluoroquinolone susceptibility clinical | 8 | 16 |
| isolate | | |
| Acinetobacter baumanii - antibiotic-susceptible clinical isolate | 64 | 16 |
| Staphylococcus aureus ATCC 29213 - antibiotic-susceptible control strain | 8 | 4 |
| Staphylococcus aureus ATCC 25923 - antibiotic-susceptible control strain | 8 | 4 |
| Staphylococcus aureus ATCC 43300 - methicillin-resistant control strain | 8 | 4 |
| Staphylococcus aureus - methicillin-resistant clinical isolate | 8 | 4 |
| Staphylococcus aureus - multi-drug-resistant clinical isolate | 16 | 4 |
| Staphylococcus aureus - teicoplanin-intermediate clinical isolate | 16 | 4 |
| Staphylococcus epidermidis antibiotic susceptible clinical isolate | 4 | 8 |
| Staphylococcus epidermidis methicillin-resistant clinical isolate | 4 | 2 |
| Staphylococcus haemolyticus - antibiotic susceptible clinical isolate | 4 | 4 |
| Staphylococcus saprophyticus - antibiotic susceptible clinical isolate | 2 | 0.5 |
| Enterococcus faecalis - ATCC 29212 antibiotic-susceptible control strain | 16 | 4 |
| Enterococcus faecalis vancomycin-susceptible clinical isolate | 32 | 4 |
| Enterococcus faecalis vancomycin-resistant (VanA) clinical isolate | 32 | 4 |
| Enterococcus faecalis vancomycin-resistant (VanB) clinical isolate | ≥128 | 8 |
| Enterococcus faecalis high-level gentamicin-resistant clinical isolate | 64 | 8 |
| Enterococcus faecium vancomycin-susceptible clinical isolate | 16 | 4 |
| Enterococcus faecium vancomycin-resistant (VanA) clinical isolate | 32 | 4 |
| Enterococcus faecium vancomycin-resistant (VanB) clinical isolate | 16 | 4 |
| Enterococcus gallinarum vancomycin-resistant (VanC) clinical isolate | 8 | 4 |
| Streptococcus pneumoniae - ATCC 49619 antibiotic-susceptible control | 32 | 16 |
| strain | | |
| Streptococcus pneumoniae - penicillin-susceptible clinical isolate | 32 | 8 |
| Streptococcus pneumoniae - penicillin-intermediate clinical isolate | 32 | 16 |
| Streptococcus pneumoniae - penicillin-resistant clinical isolate | 32 | 16 |
| Streptococcus pneumoniae - multi-drug resistant clinical isolate | 32 | 16 |
| Streptococcus pyogenes - Macrolide (MLS) resistant clinical isolate | 16 | 8 |
| Streptococcus pyogenes - Macrolide (M-type) resistance clinical isolate | 16 | 8 |
| Corynebacterium jeikeium - antibiotic-susceptible clinical isolate | 8 | 4 |
| Corynebacterium jeikeium - multi-drug resistant clinical isolate | 8 | 2 |
| Listeria monocytogenes - antibiotic-susceptible clinical isolate | 16 | 8 |
| MU50 Staphylococcus aureus (MRSA) - VISA type strain | 16 | 4 |
| EMRSA3 Staphylococcus aureus (MRSA) - SSCmec type 1 | 8 | 4 |
| EMRSA16 Staphylococcus aureus (MRSA) - SSCmec type 2 | 16 | 4 |
| EMRSA1 Staphylococcus aureus (MRSA) - SSCmec type 3 | 16 | 4 |
| EMRSA15 Staphylococcus aureus (MRSA) - SSCmec type 4 | 8 | 4 |
| HT2001254 Staphylococcus aureus (MRSA) - PVL positive | 8 | 4 |
| Streptococcus agalactiae - antibiotic-susceptible clinical isolate | 8 | 8 |
| Streptococcus agalactiae - macrolide-resistant clinical isolate | 16 | 8 |
| Group C Streptococcus - antibiotic-susceptible clinical isolate | 16 | 8 |
| Group C Streptococcus - macrolide-resistant clinical isolate | 32 | 16 |
| Group G Streptococcus - antibiotic-susceptible clinical isolate | 16 | 8 |
| Group G Streptococcus - macrolide-resistant clinical isolate | 16 | 8 |
| Streptococcus mitis - antibiotic-susceptible clinical isolate | 32 | 16 |
| Streptococcus mitis - macrolide-resistant clinical isolate | 64 | 16 |
| Streptococcus constellatus - antibiotic-susceptible clinical isolate | 64 | 16 |
| Streptococcus constellatus - macrolide-resistant clinical isolate | 32 | 16 |
| Streptococcus oralis - antibiotic-susceptible clinical isolate | 64 | 16 |
| Streptococcus oralis - macrolide-resistant clinical isolate | 64 | 16 |
| Streptococcus bovis - antibiotic-susceptible clinical isolate | 32 | 8 |
| Streptococcus bovis - macrolide-resistant clinical isolate | 8 | 2 |
| Streptococcus sanguis - antibiotic-susceptible clinical isolate | 32 | 16 |
| Streptococcus sanguis - macrolide-resistant clinical isolate | 32 | 16 |
| Clostridium perfringens - antibiotic-susceptible clinical isolate | ≥128 | 32 |
| Clostridium difficile - antibiotic-susceptible clinical isolate | 64 | 32 |
| Propionibacterium acnes- antibiotic-susceptible clinical isolate | | 4 |
| Propionibacterium acnes- antibiotic-resistant clinical isolate | | 2 |

### EXAMPLE 7 IN VIVO ACTIVITY OF COMPOUNDS 7 AND 8

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined. Results are shown in FIGS. 3, 4 and 5 expressed as the number of colony forming units per mouse.

In experiment 1 (FIG. 3), compound 7 was applied to the murine skin as part of either a cream or a gel containing 2% (w/w) of compound 7. The same cream or gel without compound 7 was used as a negative control (placebo). Bactroban 2% cream was used as a positive control. It can clearly be seen that the number of CFUs was reduced when a cream or gel containing compound 7 was applied to the murine skin, compared to the negative control, indicating that compound 7 exerted an antimicrobial effect against Staphylococcus aureus. The efficacy of standard clinical treatment, Bactroban 2% cream, had no significant effect under the treatment regime. The nature of the carrier, cream or gel, had no significant effect.

In experiment 2 (FIG. 4), compound 7 was applied in two different concentrations, as either a 1% or a 2% gel. A placebo gel and a known antibacterial “bactroban (mupericin)” were used as controls. It can be seen that gels containing compound 7 were more effective at reducing the number of CFUs from a Streptococcus pyogenes CS 301 infection than the placebo gel or the bactroban. The gel containing 2% of compound 7 was more effective than the gel containing only 1% of compound 7.

In experiment 3 (FIG. 5), compound 8 was applied in a 2% cream formulation on a Staphylococcus aureus FDA 486 infection in the murine skin infection model. A placebo cream and two known antibacterials, “Fucidin (fucidic acid) ointment 2%” and “Bactroban (mupericin) cream 2%” were used as controls. It can be seen that a cream containing compound 8 was more effective at reducing the number of CFUs than the placebo and fucidin or bactroban.

### EXAMPLE 8

8.1 Preparation of AMC-109 Compounded Silicone Sheets

A mortar (50 mm diameter) and pestle was used to grind three 0.33 g batches of AMC-109. The resulting batches were put together and ground in the mortar together for an additional 5 minutes.

Nusil™ MED-4065 (MED-4065) is a high consistency, extrusion grade silicone elastomer that is commercially available (Avantor 0). It is supplied in two parts (Part A and Part B). Part A is a mixture comprising <1% of dodecamethylcyclohexasiloxane (CAS-No. 540-97-6). Part B is a mixture comprising <5% of Siloxanes and Silicones, dimethyl, methyl hydrogen (CAS-No. 68037-59-2) and <1% of dodecamethylcyclohexasiloxane.

25 g of Part A and 25 g of Part B of MED-4065 were mixed on a two roll mill, gap 1 mm, resulting in Material “C”.

19.2 g “C” and 0.8 g of the ground AMC-109 were mixed on the two roll mill, gap 0.8 mm, until visually homogenized. The homogenized mixture was then passed 10 times, gap 0.8 mm, resulting in Material “D” containing 4% w/w AMC-109.

10 g of “D” and 10 g of “C” were mixed on the two roll mill, gap 0.8 mm, until visually homogenized. The homogenized mixture was then passed 10 times, gap 0.8 mm, resulting in Material “E” containing 2% w/w AMC-109. This dilution procedure was repeated to produce mixtures containing 1% (Material “F”) and 0.5% w/w AMC-109 (Material “G”). Each of Materials D, E, F, and G were passed 10 times, gap 0.3 mm, on the two roll mill.

From each of the materials two samples were formed using a gap of 1 mm (resulting in a sheeting of 1.5 mm thickness) and two samples were formed using of gap 0.3 mm (resulting in a sheeting of 0.5 mm thickness) respectively.

The materials were then heat cured using a platinum catalyst already present in “A” and “B” under two different curing conditions (130° C. for 4 h or 150° C. for 2 h), resulting in the samples listed in Table 7.

TABLE 7

| | | Amount of | |
|---|---|---|---|
| Sample | Curing | AMC-109 | Sheet |
| no. | conditions | (w/w %) | thickness |
| 1/19 | 130° C. 4 h | 4% | 1.5 mm |
| | 130° C. 4 h | 4% | 0.5 mm |
| 2/19 | 130° C. 4 h | 2% | 1.5 mm |
| | 130° C. 4 h | 2% | 0.5 mm |
| 3/19 | 130° C. 4 h | 1% | 1.5 mm |
| | 130° C. 4 h | 1% | 0.5 mm |
| 4/19 | 130° C. 4 h | 0.5% | 1.5 mm |
| | 130° C. 4 h | 0.5% | 0.5 mm |
| 5/19 | 150° C. 2 h | 4% | 1.5 mm |
| | 150° C. 2 h | 4% | 0.5 mm |
| 6/19 | 150° C. 2 h | 2% | 1.5 mm |
| | 150° C. 2 h | 2% | 0.5 mm |
| 7/19 | 150° C. 2 h | 1% | 1.5 mm |
| | 150° C. 2 h | 1% | 0.5 mm |
| 8/19 | 150° C. 2 h | 0.5% | 1.5 mm |
| | 150° C. 2 h | 0.5% | 0.5 mm |

8.2 Leaching of AMC-109 from Compounded Silicone Samples into Aqueous Solution

HPLC Quantitation and Standard Curve

Three analytical standards of AMC-109 for use in the quantification were prepared by dissolving 0.18 mg, 0.37 mg and 1.57 mg of AMC-109 in 5 ml of water. These were diluted prior to analysis by HPLC in the same manner as the silicone samples.

Aqueous Extraction of AMC-109 from the Compounded Silicone Samples

The compounded silicone samples were cut into samples of 500-600 mg for the 0.5 mm films and of 800-900 mg for the 1.5 mm films. The samples were further cut into 4 or 5 smaller bits before the combined cuts were placed in a vial and water (5 ml) was added. The samples were left for 40 or 42 h. A sample of 500 μl of the aqueous extract was removed and 200 μl of the HPLC solvent was added. The extraction samples were analysed by HPLC. The identity of the AMC-109 peak in the chromatograms of the compounded silicon extracts were ascertained by electrospray ionization mass spectrometry (ESI-MS).

Compounded silicone sample #1/19 (both 0.5 mm and 1.5 mm thicknesses) was selected for a second and a third extraction. Sample #1/19 was washed with water prior to the second and third extractions, which were performed as described above for the first extraction for a duration of 45 h. A sample of 500 μl was removed and 200 μl of the HPLC solvent was added. The extraction samples were analysed by HPLC.

AMC-109 was readily quantifiable by the HPLC method. The method allowed quantification over a 1 log range representing a concentration range of 40 μg/ml to 300 μg/ml. The minimum inhibitory concentration (MIC) of AMC-109 towards S. aureus and S. epidermidis is approximately 2-4 μg/ml.

The amounts of AMC-109 released from the samples in the first extraction are shown in Table 8.

TABLE 8

| | | | Amount (mg/5 ml) | |
|---|---|---|---|---|
| | Amount | | of AMC-109 | |
| | of AMC- | | in the extracts | |
| Sample | 109 | Curing | Thick film | Thin film |
| no. | (w/w %) | conditions | (1.5 mm) | (0.5 mm) |
| 1/19 | 4% | 130° C. 4 h | 0.56 | 0.70 |
| 2/19 | 2% | 130° C. 4 h | 0.20 | 0.31 |
| 3/19 | 1% | 130° C. 4 h | 0.08 | 0.13 |
| 4/19 | 0.5% | 130° C. 4 h | NQ | NQ |
| 5/19 | 4% | 150° C. 2 h | 0.67 | 0.95 |
| 6/19 | 2% | 150° C. 2 h | 0.24 | 0.30 |
| 7/19 | 1% | 150° C. 2 h | 0.11 | 0.16 |
| 8/19 | 0.5% | 150° C. 2 h | NQ | 0.11 |

*NQ denotes not quantifiable

For all but the lowest compounded concentration (0.5% w/w) the concentration in the aqueous extracts were well above the MIC of the Staphylococci. The release was highest for the thinnest films, which is in accordance with a higher surface/volume ratio for the thinner films.

The amounts of AMC-109 released from Sample #1/19 in the first, second and third extractions are shown in Table 9.

TABLE 9

| | | Amount (mg/5 ml) of | |
|---|---|---|---|
| | | AMC-109 in the extracts | |
| Sample | | Thick film | Thin film |
| no. | Extraction | (1.5 mm) | (0.5 mm) |
| 1/19 | 1st | 0.56 | 0.70 |
| | 2nd | 0.22 | 0.33 |
| | 3rd | 0.13 | 0.27 |

The results from the re-extraction experiments revealed a sustained release of AMC-109 from the silicone even after the third extraction, in total representing 6 days of sustained AMC-109 release.

The aqueous conditions that the samples were exposed to in this example mimic the in vivo environment experienced by silicone implants. The results of this example therefore show silicone materials compounded with AMP-109 would be useful for preventing bacterial contamination of silicone implants in vivo.

8.3 Microbiological Evaluation of Compounded Silicone Samples

The antimicrobial efficacy of the compounded silicone samples was assessed using an agar diffusion method.

Bacterial strains (typical Gram-positive skin bacteria):
Staphylococcus aureus (ATCC29213)
Staphylococcus epidermidis RP62a

Overnight colonies of S. aureus and S. epidermidis were diluted to 0.5 McFarland and spread on Mueller Hinton agar plates. AMC-109 compounded silicone samples: 1/19, 2/19, 3/19, 4/19, 5/19, 6/19, 7/19, and 8/19 (thickness either 0.5 mm or 1.5 mm) were rinsed and placed on newly inoculated plates two times (in total three times/sample). All experiments were performed in triplicates. Plates were incubated at 37° C. for 16 hours.

FIGS. 6 and 7 show the effect of AMC-109 compounded silicone on the growth of Staphylococcus epidermidis (FIG. 6) and Staphylococcus aureus (FIG. 7). Panel A (left) shows zones of inhibition around silicone pieces. Panel B (right) shows zones of inhibition after removal of the silicone pieces.

The size of the zone of inhibition of bacterial growth (measured with a ruler) is shown in Table 10.

TABLE 10

| | | Amount of | | | |
|---|---|---|---|---|---|
| Sample | Curing | AMC-109 | Sheet | Zone of | |
| no. | conditions | (w/w %) | thickness | inhibition | |
| 1/19 | 130° C. 4 h | 4% | 1.5 mm | 4 | mm |
| | 130° C. 4 h | 4% | 0.5 mm | 3 | mm |
| 2/19 | 130° C. 4 h | 2% | 1.5 mm | 1 | mm |
| | 130° C. 4 h | 2% | 0.5 mm | 1 | mm |
| 3/19 | 130° C. 4 h | 1% | 1.5 mm | 0.05 | mm |
| | 130° C. 4 h | 1% | 0.5 mm | 0.05 | mm |
| 4/19 | 130° C. 4 h | 0.5% | 1.5 mm | 0.05 | mm |
| | 130° C. 4 h | 0.5% | 0.5 mm | 0.5 | mm |
| 5/19 | 150° C. 2 h | 4% | 1.5 mm | 4 | mm |
| | 150° C. 2 h | 4% | 0.5 mm | 3 | mm |
| 6/19 | 150° C. 2 h | 2% | 1.5 mm | 1 | mm |
| | 150° C. 2 h | 2% | 0.5 mm | 1 | mm |
| 7/19 | 150° C. 2 h | 1% | 1.5 mm | 0.05 | mm |
| | 150° C. 2 h | 1% | 0.5 mm | 0.05 | mm |
| 8/19 | 150° C. 2 h | 0.5% | 1.5 mm | 0.05 | mm |
| | 150° C. 2 h | 0.5% | 0.5 mm | 0.05 | mm |

The silicone pieces comprising the highest amount of AMC-109 (4% w/w—sample no. 1/19 and 5/19) had the highest inhibitory zone of growth in both S. epidermidis (FIG. 6) and S. aureus (FIG. 7). The inhibitory effect was dose dependent: samples 2/19 and 6/19 (2% w/w) had a zone of inhibition of 1 mm, while samples 3/19 and 7/19 (1% w/w), and 4/19 and 8/19 (0.5% w/w) had only very small zones of inhibition. The silicone pieces seemed to release AMC in a continuous manner. In FIG. 6, panel A, top picture, one can see two clear spots, one of the spots resulted from a very brief contact with a silicone piece, which was placed on the inoculated plate for one minute only.

The silicone surfaces of sample #2/19 were investigated by fluorescence microscopy after the first exposure to the bacteria on the agar plate.

The micrographs are shown in FIGS. 8 and 9. The fluorescence micrographs reveal that there is no bacterial growth on sample #2/19 neither when exposed to S. aureus (FIG. 8) nor S. epidermidis (FIG. 9).

The samples from the agar plates were rinsed and reused twice. The reused silicone gave clear, but smaller inhibition zones than during first time use as shown in FIG. 10 (S. aureus) and FIG. 11 (S. epidermidis).

Conclusions
AMC-109 is liberated from the compounded silicone samples and provides anti-colonizing efficacy against typical skin bacteria, indicating suitability of antimicrobial compounded silicone materials for use as wound dressings.
AMC-109 is dose-dependently released from the compounded silicone.
AMC-109 compounded silicone functions as a sustained release device.
The curing procedure affects the release of AMC-109 from the compounded silicon into an aqueous solution. Higher temperatures and shorter exposure times are preferred.
Surprisingly, antimicrobial peptide compounded silicone elastomers can be cured at high temperatures of 130° C. or 150° C. without degrading the peptide.

### EXAMPLE 9

9.1 Preparation of AMC-109-Containing Silicone Pressure Sensitive Adhesive (PSA) and Polyester Films Coated with the AMC-109-Containing Silicone PSA

The following solutions of AMC-109 in ethanol were prepared

| Sample no. | Amount of AMC-109 (g) | Amount of ethanol (ml) |
|---|---|---|
| 83/19 | 0.075 | 7.5 |
| 84/19 | 0.15 | 7.5 |
| 85/19 | 0.3 | 7.5 |
| 86/19 | 0.6 | 7.5 |

Then each solution was mixed with 30 ml of silicone pressure sensitive adhesive (Nusil MED-1356, LOT: 82485, which is commercially available from Avantor and referred to herein as MED-1356) and then put on a roller mixer at room temperature. Nusil MED-1356 is a low viscosity medical grade silicone-PSA (viscosity of 250 cP [250 mPa·s] measured in accordance with standards ASTM D1084 and ASTM D2196) which contains 50% of ethyl acetate solvent. Ethyl acetate and ethanol form a stable mixture.

Solution numbers 80/19, 81/19, 87/19 and 88/19 refer to the mixtures of 30 ml MED-1356 with the solutions 83/19, 84/19, 85/19 and 86/19 respectively. After 12 hours, 80/19 and 81/19 were clear liquids whereas 87/19 and 88/19 appeared turbid and inhomogeneous. 87/19 and 88/19 were cooled to 7.5° C.±2.5° C. and put on the roll mixer overnight, which provided clear and homogeneous liquids. 80/19, 81/19, 87/19 and 88/19 were kept at 7.5° C.±2.5° C. under continuous mixing on the two roll mixer for the remainder of the study. Two control samples: 30 ml MED-1356+7.5 ml ethanol (sample no. 89/19) and pure MED-1356 (sample no. 90/19) were also prepared.

Strips of polyester film (Mylar) of thickness 0.17 mm were cut to a size of 20 mm×75 mm. The cut strips were then dipped in the AMC-109-containing silicone PSA solutions and left hanging to dry at 25° C. (i.e. cured at room temperature) to provide the following samples.

| | | | | | AMC-109 |
|---|---|---|---|---|---|
| | | Amount of | | | weight content of |
| Sample | Solution | MED-1356 | Ethanol | AMC-109 | solid matter in the |
| No. | No. | (ml) | (ml) | (g) | PSA (%*) |
| 91/19 | 80/19 | 30 | 7.5 | 0.075 | 0.5% |
| 92/19 | 81/19 | 30 | 7.5 | 0.150 | 1.0% |
| 93/19 | 87/19 | 30 | 7.5 | 0.300 | 2.0% |
| 94/19 | 88/19 | 30 | 7.5 | 0.600 | 4.0% |
| 95/19 | 89/19 | 30 | 7.5 | — | 0.0% |
| 96/19 | 90/19 | 37.5 | — | — | 0.0% |

*calculated assuming that the density of solid material in MED-1356 is 1 g/ml and that MED-1356 contains 50% by volume of ethyl acetate.

It was found that the AMC-109-containing silicone PSA samples were as sticky as the control samples (very sticky) and so AMC-109 can be incorporated into silicone PSA without affecting the stickiness of the silicone PSA.

9.2 Leaching of AMC-109 from AMC-109-Containing Silicone PSA Samples into Aqueous Solution and Microbiological Evaluation

Cuttings (approximately 1 cm) of the above polyester samples coated with AMC-109-containing silicone PSA (approximately 2 cm wide) representing a surface area of 4 cm² were placed in a vial containing 3 ml water. The cuttings were carefully shaken on an orbital shaker for 2.5 h or 24 h before samples of the aqueous extracts were collected for quantitative analysis by HPLC. The concentration of AMP-109 released into solution is shown in the table below. No additional impurity peaks were observed in the HPLC tests.

| | | Extracted | Extracted |
|---|---|---|---|
| | | concentration 2.5 h | concentration 24 h |
| Sample no. | AMC-109 | (mg/ml) | (mg/ml) |
| 91/19 (8/20) | 0.5% (EtOH) | ND | NQ |
| 92/19 (9/20) | 1% (EtOH) | NQ | 0.019 |
| 93/19 (10/20) | 2% (EtOH) | 0.026 | 0.0389 |
| 94/19 (11/20) | 4% (EtOH) | 0.030 | 0.0815 |
| 95/19 (12/20) | 0% (EtOH) | ND | ND |
| 96/19 (13/20) | 0% | ND | ND |

*ND denotes not detected, NQ denotes not quantifiable (but detected)

For the microbiological evaluation, overnight colonies of S. epidermidis were used to make 0.5 McFarland (1×10⁸ CFU/ml) solutions in 0.5% NaCl and further diluted to 105 CFU/ml. 150 μl of bacterial solution was applied to the polyester samples coated with AMC-109-containing silicone PSA (0.5 mm×10 mm), placed in an incubation chamber and incubated at 37° C. for 18 hours. The material was resuspended in 5 ml NaCl, vortexed for 20 seconds and used for making serial dilutions (10⁻¹ to 10⁻⁶) in 1 ml NaCl. From the different dilutions, 100 μl was spread on blood agar plates and further incubated at 37° C. for 18 hours. The number of CFU was then assessed. All experiments were repeated two times, except for sample 92/19 the experiment was repeated four times. The results are shown in the table below. No bacteria were observed after the bacterial solution was applied to samples 92/19, 93/19 or 94/19.

| Sample | AMC-109 | CFU |
|---|---|---|
| 92/19 (9/20) | 1% (EtOH) | 0 |
| 93/19 (10/20) | 2% (EtOH) | 0 |
| 94/19 (11/20) | 4% (EtOH) | 0 |
| 95/19 (12/20) | 0% (EtOH) | 2.2 × 10⁷ |
| 96/19 (13/20) | 0% | 1.8 × 10⁷ |

Conclusions
AMC-109 can be incorporated into medical grade silicone PSA using standard techniques without adversely affecting the adherent characteristics of the silicone PSA.
AMC-109 is liberated from the silicon samples and provides anti-colonizing efficacy against typical skin bacteria, indicating suitability for use as an antimicrobial adhesive, for example for fixing medical devices (e.g. bandages) to skin.
AMC-109 is dose-dependently released from the AMC-109-containing silicone PSA.
The samples showed continued released of AMC-109 over at least 24 h. AMC-109-containing silicone PSA can therefore function as a sustained release device.

### EXAMPLE 10

10.1 Incorporation of AMC-109 into Medical Grade Silicone by a “Swell and Dry” Method

Parts A and B of MED-4065 (as described in Example 8) were mixed in a 1:1 wt. ratio and processed on a two roll mill (gap 0.8 mm), resulting in sheets of 1 mm thickness. The heat cured (1 hour at 120° C.) sheets were cut to provide silicone samples of dimensions 2×1 cm.

Swelling agents were produced by mixing:

0.18 g AMC-109 in 2 ml ethanol and 4 ml chloroform resulting in a 2.3 wt. % solution, or

0.09 g AMC-109 in 2 ml ethanol and 4 ml chloroform resulting in a 1.15 wt. % solution.

The silicone samples were divided into 4 pieces and soaked for various exposure times resulting in different absorptions with the aim of achieving absorption percentages of 5%, 10%, 20%, 50%, and 100%. After soaking, the samples were superficially dried with cellulose wipes, weighed, and then dried at 20° C. for 12 hours.

Sample numbers, the concentration of AMC-109 in the swelling agent, exposure time, initial weight (total of 4 pieces per sample), weight after exposure, and absorption percentage [100%×(weight after swelling−initial weight)/initial weight] are shown in the table below.

| | Concentration | | | Weight | |
|---|---|---|---|---|---|
| | of AMC-109 | | Initial | after | |
| | in swelling | Exposure | weight | swelling | % |
| Sample no. | agent (wt. %) | time | (g) | (g) | absorption |
| 19/20 | 2.3% | 10 | 1.072 | 1.122 | 4.7% |
| | | seconds | | | |
| 20/20 | 2.3% | 30 | 1.078 | 1.174 | 8.9% |
| | | seconds | | | |
| 21/20 | 2.3% | 120 | 1.152 | 1.375 | 19.4% |
| | | seconds | | | |
| 22/20 | 2.3% | 15 minutes | 1.103 | 1.77 | 60.5% |
| 25/20 | 2.3% | 120 | 1.137 | 2.022 | 77.8% |
| | | minutes | | | |
| 29/20 | 1.15% | 10 | 1.064 | 1.113 | 4.6% |
| | | seconds | | | |
| 30/20 | 1.15% | 30 | 1.114 | 1.22 | 9.5% |
| | | seconds | | | |
| 31/20 | 1.15% | 120 | 1.136 | 1.35 | 18.8% |
| | | seconds | | | |
| 32/20 | 1.15% | 15 minutes | 1.083 | 1.649 | 52.3% |
| 28/20 | 1.15% | 120 | 1.12 | 2.132 | 90.4% |
| | | minutes | | | |
| 34/20 | 0.0% | 120 | 1.108 | 2.173 | 96.1% |
| (control)* | | minutes | | | |
| 35/20 | | | | | |
| (control)** | | | | | |

*sample no. 35/20 is a control sample which was exposed to swelling agent not comprising AMC-109
**sample no. 35/20 is a control sample which was not exposed to swelling agent

Without wishing to be bound by theory, it is believed that during the swelling process a concentration gradient is present with the outer layer of the material being saturated whereas the inner layers are only partially saturated. By breaking off the swelling process before saturation of the whole sample is reached, a product can be obtained in which the surface layers contain the desired concentration of peptide whilst the inner layers contain less peptide. This enables more cost-effective preparation of antimicrobial articles.

10.2 Leaching of AMC-109 into Aqueous Solution from the Silicone Samples Loaded with AMC-109 by the Swell and Dry Method and Microbiological Evaluation of these Samples

Extraction

The silicone samples prepared using the “swell and dry” method were cut into approximately equally sized parts. The cut samples were placed in a vial and water (1 ml) was added. The samples were left for 1.5 h. The extraction samples were then filtered and analysed by HPLC. The results are shown in the table below.

| | AMC-109 concentration | | Concentration in |
|---|---|---|---|
| | in swelling agent | | aqueous extract |
| Sample No. | (wt. %) | Swell time | (mg/ml) |
| 19/20 | 2.3% | 10 seconds | 0.023 |
| 20/20 | 2.3% | 30 seconds | 0.011 |
| 21/20 | 2.3% | 120 seconds | 0.012 |
| 22/20 | 2.3% | 15 minutes | 0.024 |
| 25/20 | 2.3% | 120 minutes | 0.049 |
| 29/20 | 1.15% | 10 seconds | 0.005 |
| 30/20 | 1.15% | 30 seconds | 0.007 |
| 31/20 | 1.15% | 120 seconds | 0.009 |
| 32/20 | 1.15% | 15 minutes | 0.11 |
| 28/20 | 1.15% | 120 minutes | 0.014 |
| 34/20 (control) | 0.0% | 120 minutes | ND |
| 35/20 (control) | No swelling | | ND |

Microbiological Assay

Overnight colonies of S. epidermidis (RP62A) were diluted to 0.5 McFarland (1×10⁸ CFU/ml) in 0.85% NaCl. The bacterial solution was further diluted in Tryptic Soy Broth media (to 1×10⁵ CFU/ml) and drops of 100 μl were applied to the surfaces of the silicone samples. The silicone samples were placed on glass microscope slides, which were then placed in a moist incubation chamber and incubated for 24 hours at 37° C.

For determining CFU values, the samples were vortexed in 2 ml 0.85% NaCl and serial dilutions (10⁻¹ to 10⁻⁶) were made. 100 μl aliquots of the serial dilutions were streaked on blood agar plates, and further incubated overnight, prior to CFU counting. All experiments were performed twice. The results are shown in the table below.

| | Swelling agent | | | |
|---|---|---|---|---|
| | AMC-1109 | | | No. of CFU |
| Sample no. | content | Exposure time | | (CFU/ml) |
| 21/20 | 2.3% | 120 | s | 0 |
| 22/20 | 2.3% | 15 | min | 0 |
| 25/20 | 2.3% | 120 | min | 0 |
| 29/20 | 1.15% | 10 | s | 0 |
| 30/20 | 1.15% | 30 | s | 0 |
| 31/20 | 1.15% | 120 | s | 0 |
| 32/20 | 1.15% | 15 | min | 0 |
| 28/20 | 1.15% | 120 | min | 0 |
| 34/20 Control | 0.0% | 10 | s | 3.3 × 10⁸ |

| | | | |
|---|---|---|---|
| 35/20 Control | No swelling | — | 9 × 10⁸ |

The level of bacterial inhibition was examined by number of CFU. The material showed good antimicrobial effect. All control samples showed a high level of bacterial surface colonisation.

Conclusions
AMC-109 can be reversibly loaded into medical grade silicon using a swell and dry method.
AMC-109 will be liberated from the samples and provide anti-colonizing and antimicrobial activity in the local environment around the silicone article as well as on the surface and interior of the silicone article.
The released amount of AMC-109 is generally dependent on the amount of active ingredient in the swelling solvent and the swell time

### EXAMPLE 11

11.1 Incorporation of AMC-109 into a Commercially Available Silicone Prosthetic Liner by a “Swell and Dry” Method

Samples were cut using a scalpel from a silicone prosthetic liner (Iceross® Original Locking under limb prosthesis) that is commercially available from the Icelandic company Össur. The samples were cut into rectangular pieces (2 cm×1 cm) and had a thickness of approximately 2 mm. The swelling was performed using a mixture containing 100 mg of AMC-109 in 4 ml of 2-propanol and 8 ml of pentane. The dissolution of AMC-109 in 2-propanol is rather slow and 2-3 h is required to obtain a clear mixture. Three swell times were used, 5 min, 15 min and 30 min. To correct for AMC-109 deposited on the surface, and not penetrated into the material, a series of samples were quickly washed with water before subsequent analysis. The table below shows the sample number, swell time and an indication of whether the sample was quickly washed with water before subsequent analysis.

| Sample no. | Swell time | Aqueous washing |
|---|---|---|
| WS-070 | 5 minutes | Yes |
| WS-072 | 5 minutes | No |
| WS-074 | 15 minutes | Yes |
| WS-069 | 30 minutes | Yes |
| WS-071 | 30 minutes | No |

11.2 Leaching of AMC-109 into Aqueous Solution from the Silicone Samples

One sample of each preparation was analysed with respect to aqueous extraction from the specimen. The silicone samples were cut in two approximately equally sized parts that were placed in a vial and water (1 ml) was added. The samples were left for 0.5 hours. The extraction samples were filtered and analysed by HPLC using UV-detection at 280 nm and quantified using a standard curve. The table below shows the concentration of AMC-109 in the extracts.

| Sample no. | Swell time | Aqueous washing | Concentration (mg/ml) |
|---|---|---|---|
| WS-070 | 5 minutes | Yes | 0.03 |
| WS-072 | 5 minutes | No | 0.29 |
| WS-074 | 15 minutes | Yes | 0.05 |
| WS-069 | 30 minutes | Yes | 0.11 |
| WS-071 | 30 minutes | No | 0.53 |

The results from the aqueous extraction shows a clear correlation between the swell time and the amount of extractable AMC-109. The results also show that a relatively large amount of AMC-109 resides on the surface, and is readily removable by a quick water wash, but also that a sizable amount is absorbed by the silicone material. The amount of absorbed, but extractable AMC-109 would provide an effective antimicrobial environment in the vicinity of the product.

11.3 Microbiological Evaluation

Bacterial Strains:
Staphylococcus epidermidis RP62A
Staphylococcus hominis 58-69

Overnight colonies of S. epidermidis or S. hominis were diluted to 0.5 McFarland (1×10⁸ CFU/ml) in 0.85% NaCl. The bacterial solution was further diluted in Tryptic Soy Broth media (to 1×10⁵ CFU/ml) and drops of 100 μl was applied to the surfaces of the silicone patches. Silicone patches were placed on glass microscope slides, and the glass slides were placed in a moist incubation chamber and incubated for 24 hours at 37° C.

For determining CFU, the samples were vortexed in 1 ml 0.85% NaCl and serial dilutions (10⁻¹ to 10⁻⁶) were made. 100 μl aliquots of the serial dilutions were streaked on blood agar plates, and further incubated overnight prior to CFU counting.

The level of bacterial inhibition was examined by number of CFU and growth zone inhibition. All materials tested were washed with water after completion of the swell and dry procedure to ensure that the activity of the materials was due to internally absorbed AMC-109 only.

The table below shows the swelling time and number of CFU for bacteria grown on the silicone surfaces. Silicone swollen for both 5 and 15 minutes showed excellent antimicrobial efficiency compared to control samples (silicone swollen without AMC-109).

| | 5 minutes swell | 15 minutes swell | |
|---|---|---|---|
| | time | time | Control |
| Bacterial strain | CFU/ml | CFU/ml | CFU/ml |
| S. epidermidis | 0 | 0 | 1.4 × 10⁸ |
| S. hominis | 0 | 0 | 1.8 × 10⁷ |

The table below shows the size of the growth inhibition zones around the silicone surfaces. The growth inhibition zones did not differ between the two samples with different swelling times in which the swelling agent contained AMC-109. The control samples (silicone swollen without AMC-109) showed no growth zone inhibition

| | 5 minutes swell | 15 minutes swell | |
|---|---|---|---|
| Bacterial strain | time | time | Control |
| S. epidermidis | 3 mm | 3 mm | 0 |
| S. hominis | 4 mm | 2 mm | 0 |

Conclusions
AMC-109 can be compounded into silicon prosthetic liners using a swell and dry method.
A swelling agent comprising a 2-propanol/pentane solvent mixture is suitable for providing reversible swelling. Reversible swelling is desirable to avoid deformation of the impregnated material but still give deposition of the active agent within the material.
AMC-109 was liberated from the samples and provided anti-colonizing and local antimicrobial efficacy against both S. aureus and S. hominis (the latter responsible for much of the unpleasant smell associated with silicone liners).
The released amount of AMC-109 is dependent on the swell time.

### EXAMPLE 12

12.1 Incorporation of AMC-109 into Silicone Foam Wound Dressing Adsorbents

Preparation of AMC-109 Containing Silicone Wound Dressings

Material: Mepilex Lite (Mölnlycke Healthcare)
Allevyn Gentle border (Smith & Nephew)

The silicone foam absorbent from commercial dressings were cut into 2 cm×2 cm square patches using scissors. Ethanolic solutions (400 μl) with varying amounts of AMC-109 were added dropwise to evenly cover the surface of the cut patches before drying in a fume hood for 72 hours. Control samples of the absorbent dressings were made similarly using ethanol only.

12.2 Microbiological Analysis

Modified AA TCC 100 Method

Overnight colonies of S. aureus were diluted to 0.5 McFarland (1×10⁸ CFU/ml) in 0.9% NaCl and further diluted to 10⁵ CFU/ml in Tryptic Soy Broth media (TSB). The different silicone samples were inoculated with 400 μl of the bacterial solution and incubated at 37° C. for 24 hours in an incubation chamber. After incubation the material was vortexed in 4 ml 0.9% NaCl for 20 seconds and used for making serial dilutions (10⁻¹-10⁻⁶) in 0.9% NaCl. From the different dilutions, 100 μl was spread on tryptic soy agar plates and further incubated at 37° C. for 24 hours. Three biological replicates of each test material were made.

Results

Colony forming counts for S. aureus (median data shown).

| | Colony forming units | | | |
|---|---|---|---|---|
| | | 0.05% AMC- | 0.2% AMC- | 0.4% AMC- |
| | 0% AMC-109 | 109 | 109 | 109 |
| Mepilex Lite | 4.5 × 10⁸ | 3.5 × 10² | 0 | 0 |
| Allevyn gentle | 1.9 × 10⁸ | 0 | 0 | 0 |
| border | | | | |

Conclusions
Silicone foam can be impregnated with AMC-109
AMC-109 was liberated from the samples and provided antimicrobial efficacy against S. aureus.

### EXAMPLE 13

13.1 Incorporation of AMC-109 into Solid Silicone by a “Swell and Dry” Method

Silicone Membrane Samples: Cylindrical pieces (8 mm diameter, 6 mm height) composed of medical grade silicone (solid silicone membrane valves produced by Tada Medical AB).

Swelling & Drying

The swelling solvent used was a 2:1 (v) mixture of 2-propanol and pentane. Four rounds of swelling were performed:
1. High loading—Silicone valves submerged in swelling solvent for 30 min with and without AMC-109 (1 mg/0.120 ml)
2. Low loading—Silicone valves submerged in swelling solvent for 30 min with and without AMC-109 (0.2 mg/0.120 ml)
3. Low loading with washing—Silicone valves submerged in swelling solvent for 30 min with AMC-109 (0.375 mg/0.120 ml). The valves were dried (1 h) and washed (dipped in 2-propanol) and dried (48 h). Control material was treated analogously in swelling solvent without AMC-109.
4. Low loading with expanded bacterial panel—Silicone valves submerged in swelling solvent for 30 min with and without AMC-109 (0.2 mg/0.120 ml). Tested against S. epidermidis (RP62a), E. coli (5067-6002) and P. aeruginosa (PAO1).

13.2 Microbiological Assay

Modified AA TCC 100 Method

Overnight colonies of S. aureus, S. epidermidis, E. coli and P. aeruginosa were diluted to 0.5 McFarland in 0.85% NaCl resulting in a bacterial concentration of 1×10⁸ bacteria. This solution was further diluted in TSB to 1×10⁵ bacteria.

After being compounded with AMC-109 by the swell and dry method, TADA silicone material was cut in pieces of 0.4 cm, the diameter of the piece was 0.7 cm, the pieces were then vertically sliced in two halves. Both sliced and unsliced samples were tested. The different samples were inoculated with 50 μl of the bacterial solution (1×10⁵) and incubated at 37° C. for 24 hours. Bacterial solution was applied to an original surface of the material or onto a surface created by slicing the material. Three biological replicates of each test material were made.

After incubation the silicone material was placed in 500 μl NaCl and vortexed for 20 seconds, before making serial dilutions (0-10⁻⁶) and plating of 100 μl for CFU counting.

13.3 Results

Swelling Efficacy

The swelling was performed as described and the material swelled to a substantial degree (by visual assessment—the base of the valve increased in diameter by 10-20%). This linear swelling of approximately 15% represents a volume increase of approximately 50%.

After drying the silicone membrane samples regained its original size (by visual inspection).

Microbiological Efficacy

The number of CFU was zero for all material containing AMC-109., compared to the control material where the CFU was determined to be in the range of 10⁴ to 10⁸, as shown in the table below.

S. aureus (ATCC 8325) CFU values for silicone membrane samples impregnated with AMC-109.

| | | Surface | CFU | |
|---|---|---|---|---|
| | | inoculated | (samples | |
| AMC-109 | Swell | with bacterial | containing | CFU |
| loading | concentration | solution | AMC-109) | (Control) |
| High | 8.3 mg/ml | A* | 0 | 2.53 × 10⁸ |
| Low with | 3.1 mg/ml | A* | 0 | 2.7 × 10⁶ |
| washing | | | | |
| Low with | 3.1 mg/ml | B* | 0 | 8.3 × 10⁴ |
| washing | | | | |
| Low | 1.7 mg/ml | B* | 0 | 4.7 × 10⁵, |

A* = an original surface of the material
B* = a surface created by slicing the material

S. epidermidis (RP62a), E. coli (5067-6002) and P. aeruginosa (PAO1) CFU values for silicone membrane samples impregnated with AMC-109 (low loading).

| | Surface | | |
|---|---|---|---|
| | inoculated with | CFU (samples | |
| | bacterial | containing AMC- | |
| | solution | 109) | CFU (Control) |
| P. aeruginosa | A* | 0 | 2.8 × 10⁸ |
| E. coli | A* | 0 | 9.6 × 10⁷ |
| S. epidermidis | A* | 0 | 3.5 × 10⁸ |

A* = an original surface of the material

All material swelled in the presence of AMC-109, showed no colonization and complete bacterial killing. Two approaches were used to eliminate the AMC-109 deposited on the surface of the material (i.e. not truly integrated); either washing with 2-propanol or by cutting and exposing the inner surface only. Either technique, either alone or in concert, proved complete antimicrobial efficacy.

Conclusions
AMC-109 can be impregnated into solid silicone products using a swell and dry method.
The local antimicrobial efficacy is complete for S. aureus (ATCC 8325), S. epidermidis (RP62a), E. coli (5067-6002) and P. aeruginosa (PAO1).
The results from the wash and cut experiments shows that AMC-109 is impregnated into the interior of the product, but the product still shows very good anti-colonizing and local antimicrobial efficacy.

1-23. (canceled)

24. A formulation comprising a silicone substrate which comprises a compound of formula (I)
AA-AA-AA-X—Y (I)
wherein, in any order, 2 of said AA (amino acid) moieties are cationic amino acids and 1 of said AA is an amino acid with a lipophilic R group, the R group having 14-27 non-hydrogen atoms;
X is a N atom, which may be substituted by a branched or unbranched C1-C10 alkyl or aryl group, which group may incorporate up to 2 heteroatoms selected from N, O and S; and
Y is selected from the group consisting of R1-R2-R3,
R1-R2-R2-R3, R2-R2-R1-R3, R1-R3, and R4
wherein:
R1 is C, O, S or N,
R2 is C;
each of R1 and R2 may be substituted by C1-C4 alkyl groups or unsubstituted;
R3 is a group comprising 1 to 3 cyclic groups each of 5 or 6 non-hydrogen atoms, 2 or more of the cyclic groups may be fused and one or more of the cyclic groups may be substituted; R3 incorporates a maximum of 15 non-hydrogen atoms; and
R4 is an aliphatic moiety having 2-20 non-hydrogen atoms, said moiety being linear branched or cyclic;
wherein the compound of formula (I) is capable of being released from the formulation.

25. The formulation of claim 24, wherein said compound is a peptide.

26. The formulation of claim 24, wherein said compound is a compound of formula (II)
AA1-AA2-AA1-X—Y (II)
wherein:
AA1 is a cationic amino acid;
AA2 is an amino acid with a lipophilic R group, the R group having 14-27 non-hydrogen atoms; and
X and Y are as defined in claim 24.

27. The formulation of claim 24, wherein said compound has the structural formula

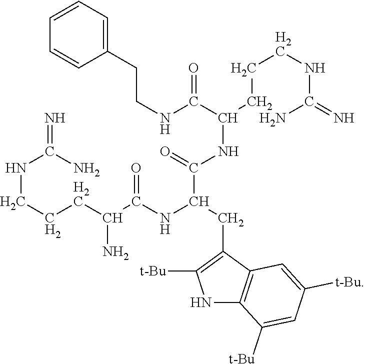 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

28. The formulation of claim 24, wherein said compound is releasably dispersed through all or a part of the silicone substrate.

29. The formulation of claim 24 wherein the silicone is medical grade silicone, and/or wherein the silicone substrate is a rubber, gel, fluid, adhesive, sealant, foam, sheet, coating or membrane.

30. A medical device comprising or consisting of a formulation of claim 24.

31. The device of claim 30, wherein said medical device is a wound dressing, indwelling device such as a catheter or valve, dermal patch, adhesive, contact lens, breast or other implant or a liner for a prosthesis.

32. The device of claim 31, wherein said adhesive is a pressure sensitive adhesive.

33. A method of producing a formulation comprising a silicone substrate compounded with a compound of formula (I) as defined in claim 24, which method comprises curing a curable silicone elastomer composition into which a compound of formula (I) has been mixed to provide the silicone substrate compounded with the compound of formula (I).

34. The method of claim 33, wherein the curable silicone elastomer composition into which a compound of formula (I) has been mixed is cured in the presence of a catalyst.

35. The method of claim 34, wherein the catalyst is a platinum catalyst.

36. The method of claim 33, wherein the curing comprises heating to a temperature in the range of 100-200° C.

37. The method of claim 33, wherein the duration of the curing is 1-5 hours.

38. The method of claim 33, wherein the compound of formula (I) has the structure

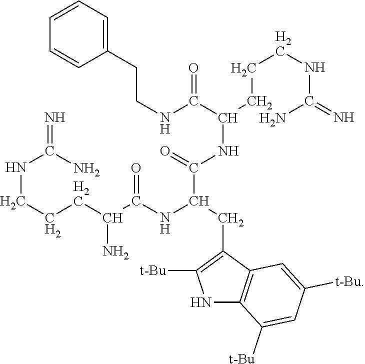 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

39. A method of producing a formulation as defined in claim 33, said method comprising (i) preparing a solution of one or more solvents and a compound of formula (I) dissolved therein, and (ii) applying said solution to a silicone substrate.

40. The method of claim 39 wherein (A) the product of step (ii) is cured and/or dried; and/or (B) the solution comprises two solvents.

41. The method of claim 39 which method comprises dissolving a compound of formula (I) in a first solvent and then mixing it with a silicone containing formulation which also comprises a solvent which is the same as or miscible with said first solvent.

42. The method of claim 41 wherein the resultant formulation is a silicone adhesive.

43. A method of treating or preventing an infection which method comprises applying to a subject in need thereof a therapeutically effective amount of a formulation as defined in claim 24.
